(19) United States
(12) Patent Application Publication
(10) Pub. No.: US 2013/0085101 A1
(43) Pub. Date: Apr. 4, 2013

(54) LONG-ACTING INSULIN ANALOGUE PREPARATIONS IN SOLUBLE AND CRYSTALLINE FORMS

(71) Applicant: Michael Weiss, Moreland Hills, OH (US)

(72) Inventor: Michael Weiss, Moreland Hills, OH (US)

(73) Assignee: CASE WESTERN RESERVE UNIVERSITY, Cleveland, OH (US)

(21) Appl. No.: 13/580,656

(22) PCT Filed: Feb. 22, 2011
(86) PCT No.: PCT/US11/25730

§ 371 Date: Nov. 13, 2012

Related U.S. Application Data
(60) Provisional application No. 61/306,722, filed on Feb. 22, 2010.

(51) Int. Cl. A61K 38/28

(52) U.S. Cl. CPC A61K 38/28
USPC 514/6.2; 536/23.51; 435/320.1; 435/252.33; 435/254.21; 435/254.23

(57) ABSTRACT

A pharmaceutical formulation comprises an insulin analogue or a physiologically acceptable salt thereof, wherein the insulin analogue or a physiologically acceptable salt thereof contains an insulin A-chain sequence that contains paired Histidine substitutions at A4 and A8, and optionally a substitution at A21. The formulation further contains a pharmaceutically acceptable buffer containing at least about 4 zinc ions per 6 insulin analogue molecules. The formulation forms a long-acting zinc-dependent subcutaneous depot upon subcutaneous injection. In a zinc-free formulation, the insulin analogue monomer exhibits decreased affinity for the Insulin-like Growth Factor receptor and at least 20% of the affinity for the insulin receptor of the same species, in comparison to an otherwise identical insulin or insulin analogue that does not contain the His^{A4} and His^{A8} substitutions.

### CROSS REFERENCE TO RELATED APPLICATIONS

This application is a national stage application of PCT/US2011/0025730 which was filed on Feb. 22, 2011, which claims priority from U.S. Provisional Application No. 61/306,722 filed on Feb. 22, 2010.

### STATEMENT REGARDING FEDERALLY SPONSORED RESEARCH OR DEVELOPMENT

This invention was made with government support under cooperative agreements awarded by the National Institutes of Health, Contract Nos. NIH R01 DK40949, RO1DK069764 and R01-DK74176. The U.S. government may have certain rights to the invention.

### BACKGROUND OF THE INVENTION

Intensive insulin therapy for the treatment of Type 1 diabetes mellitus requires subcutaneous injection of an insulin formulation or of an insulin analogue formulation. Regimens may consist of multiple daily injections or continuous subcutaneous infusion of insulin or of an insulin analogue (“pump therapy”). Control of blood glucose concentrations is sought during, after, and between meals and through the sleep-wake cycle. Because pumps enabling continuous infusion are used by only a minority of patients, considerable efforts have been undertaken to develop short-, intermediate-, and long-acting formulations, which are typically defined as human insulin preparations, mammalian insulin preparations, or insulin analogue preparations, with effective-durations of approximately 4, 12, and 18-24 hours, but potentially lasting up to 7, 16, and 30 hours respectively. Particular interest in long-acting insulin formulations, or long-acting insulin analogue formulations, has been motivated by the need to avoid nocturnal hypoglycemia and/or morning hyperglycemia. The present invention pertains to methods of preparation of a novel class of long-acting insulin analogue formulations. This class of formulations may also be useful for the treatment of Type 2 diabetes mellitus.

Administration of insulin is long established as a treatment for diabetes mellitus. Insulin is a small globular protein that plays a central role in metabolism in vertebrates. Insulin contains two chains, an A-chain, containing 21 residues and a B-chain containing 30 residues. The hormone is stored in the pancreatic β-cell as a Zn²⁺-stabilized hexamer, but functions as a Zn²⁺-free monomer in the bloodstream.

Insulin is the product of a single-chain precursor, proinsulin, in which a connecting region (35 residues) links the C-terminal residue of B-chain (residue B30) to the N-terminal residue of the A-chain (FIG. 1A). The structure of proinsulin, as recently determined by nuclear magnetic resonance as an engineered monomer, contains an insulin-like core and disordered connecting peptide as long envisaged (FIG. 1B). Formation of three specific disulfide bridges (A6-A11, A7-B7, and A20-B19; FIG. 1B) is thought to be coupled to oxidative folding of proinsulin in the rough endoplasmic reticulum (ER). Proinsulin assembles to form soluble Zn²⁺-coordinated hexamers shortly after export from ER to the Golgi apparatus. Endoproteolytic digestion and conversion to insulin occurs in immature secretory granules followed by morphological condensation. Crystals of zinc-insulin hexamers within mature storage granules have been visualized by electron microscopy (EM). The present invention describes insulin analogue formulations that direct novel zinc-containing multi-hexamer assemblies to modify the duration of action of an active insulin analogue on subcutaneous injection.

Design of insulin analogues in use for the treatment of diabetes mellitus has exploited the three-dimensional structure of insulin as a monomer, dimer, and hexamer. An insulin monomer contains three α-helices, two β-turns, and two extended segments. The A-chain consists of an N-terminal α-helix (residues A1-A8), non-canonical turn (A9-A12), second α-helix (A12-A18), and C-terminal extension (A19-A21). The B-chain contains an N-terminal arm (B1-B6), β-turn (B7-B10), central α-helix (B9-B19), β-turn (B20-B23), β-strand (B24-B28), and flexible C-terminal residues B29-B30. The two chains pack to form a compact globular domain stabilized by three disulfide bridges (cystines A6-A11, A7-B7, and A20-B19). Extensive X-ray crystallographic studies have been undertaken of the Zn²⁺-coordinated insulin hexamer in a variety of lattice forms; the multiple crystal forms define three structural families designated T₆, T₃R^{f}₃ and R₆. In each case, it has been found that two Zn ions lie along the central axis of the hexamer (“axial zinc ions”), each coordinated by three Histidine side chains (His^{B10}); additional low-affinity or partially occupied zinc-binding sites have been observed in some crystal forms. The T-state protomer resembles the structure of an insulin monomer in solution. The R-state protomer exhibits a change in the secondary structure of the B chain: the central α-helix extends to B1 (the R state) or to B3 (the frayed R^{f} state). The three families of hexamers also differ in subtle features of side-chain packing.

Subcutaneous disassembly of insulin hexamers can be a key driver of injected insulin pharmacokinetics. Hence, pharmaceutical insulin formulations have often been based on assembly or disassembly of zinc insulin hexamers. For example, rapid-acting analogues may limit insulin hexamer self-assembly or accelerate hexamer disassembly. On the other hand, long-acting analogues typically retard disassembly or promote precipitation and self-assembly in a subcutaneous depot. Pertinent to the design and pharmacokinetics of Humalog® and NovoLog®, for example, the constituent insulin analogues are injected as hexamers, which must disassemble to permit absorption into the capillaries. Substitutions in those analogues facilitate hexamer disassembly to enable a fast-acting insulin formulation. In contrast, long-acting Lantus® is injected as a solution of primarily monomers and dimers, which precipitate to form an amorphous or microcrystalline depot after injection as the pH is raised in the injectate on buffering by subcutaneous tissue and fluids. These strategies depend on a common principle—a relationship between the availability of free insulin monomers or dimers in the subcutaneous depot and its rate of absorption into capillaries. A variety of insulin formulations so developed provides a range of pharmacokinetic properties. A combination of short-acting, intermediate, and long-acting insulin formulations or insulin analogue formulations enables design of a daily regimen to constrain fluctuations in blood glucose concentration and hence optimize glycemic control. The major classes of clinical formulations are:
Regular Insulin—Rapid-acting insulin formulations are formulated at neutral pH as clear solutions of soluble zinc insulin hexamers. Phenol, meta-cresol, or methylparaben, originally introduced as antimicrobial preservatives, also bind to the hexamers to induce the TR structural transition. The R₆ hexamer exhibits higher thermodynamic and kinetic stability than the classical T₆ hexamer. Analogous zinc-based hexameric insulin analogue formulations are used for rapid-acting products Humalog® (Eli Lilly and Co.) and Novolog® (Novo-Nordisk).
NPH Insulin—Intermediate-acting insulin formulations (NPH; neutral protamine Hagedorn) are based on suspensions of orthorhombic crystals of R₆ zinc insulin hexamers containing phenol (or meta-cresol) and sub-stoichiometric concentrations of protamine, mixture of small basic peptides containing multiple arginine residues. X-ray crystallographic studies of NPH insulin crystals suggest that the positions of these basic peptides in the crystal their modes of binding to the zinc insulin hexamer. An analogous NPH formulation of the otherwise-rapid acting insulin lispro (the active component of Humalog®) has been developed to allow mixed regimens. However, NPH insulin is difficult and expensive to formulate: protamine is a collection of basic peptides derived from sperm, usually beef sperm; producing NPH crystals is an exacting and complex process built around an initial production of uniform seed crystals. Furthermore, NPH insulin is prone to fibrillation.
The Lente Principle—Also designated insulin zinc suspensions (IZS), protracted action by human insulin or animal insulins may be obtained by adding an excess of zinc ions (typically 20-30 per hexamer) to suspensions of T₆ insulin hexamers. This large excess leads to binding low-affinity sites and yields amorphous precipitates of zinc insulin complexes (Semilente or IZS amorphous) or rhombohedral zinc T₆ insulin micro-crystalline suspensions (Ultralente or IZS, crystalline). Methylparaben is typically used as preservative and binds to one face of the T₆ zinc insulin hexamer. Ultralente formulations are more long acting than Semi-lente formulations; intermediate time courses can be obtained by mixing amorphous and crystalline particles (Lente or IZS, mixed). The following two steps are undertaken in manufacture:
(1) Precursor Insulin Crystals—The first step employs zinc ions and a high (supra-physiological) concentration of chloride ions (1.2 M NaCl) in the absence of preservative to form a suspension of micro-crystalline seeds at pH 5.5. The precursor crystals belong to space group R3 and contain T₃R^{f}₃ zinc insulin hexamers in which a total of four zinc ions (+8 in charge) and seven chloride ions (−7 in charge) are bound per hexamer, together providing +1 to the formal charge of the hexamer. Unlike the R₆ hexamers of regular formulations, the precursor hexamers contain only one axial zinc ion, located within the T₆ trimer. The other three zinc ions are off-axis within the R^{f}₃ trimer: His^{B10} flips its conformation in concert with His^{B5} and two chloride ions to form a tetrahedral zinc-ion-binding site. These off-axis sites are near the phenol-binding pockets of classical R₆ hexamers. The intrahexamer, off-axis zinc-ion-binding sites of ultralente precursor crystals are unrelated to the interfacial/interhexamer zinc-binding sites of the present invention.
(2) Ultralente Insulin Crystals—To obtain the ultra-lente microcrystalline suspension, the seed crystals are diluted into a buffer at pH 7.4 containing methylparaben, a lower concentration of chloride ions (120 mM), and higher concentration of zinc ions. The crystals consist of T₆ insulin hexamers in space group R3. As a result of the very high zinc ion concentrations in the formulation (e.g., >5 per insulin molecule), extra zinc ions are observed per hexamer. In addition to the normal two axial zinc ions, there is observed partial occupancy of one of two mutually exclusive non-classical, weak-binding sites also located in the center of the hexamer. The electron density was not of sufficient quality to allow analysis of bound chloride ions. The intrahexamer, off-axis zinc-ion-binding sites of mature ultralente crystals are also unrelated to the interfacial/interhexamer zinc-binding sites of the present invention.
Insulin analogues with extended B-chains that form hexamers with more than 2 zinc per hexamer are also known. Human insulin with the following substitution sets: GlyA21-HisB31-HisB32, GlyA21-HisB31-HisB32-ArgB33, GlyA21-AlaB31-HisB32-HisB33, and GlyA21-AlaB31-HisB32-HisB33-ArgB34, form stable complexes with 6.5, 5.3, 6.7, and 5 zinc per hexamer respectively. These complexes (GlyA21-AlaB31-HisB32-HisB33-ArgB34 in particular) have also shown extended pharmacokinetics in dogs. It is likely here that the extra zinc ions are binding between the alpha-amino groups of A-chain and the new Histidines at the C-terminus of the B-chain within each hexamer.
Other—Protracted action has been achieved following subcutaneous injection of a clear acidic solution of an insulin analogue (insulin glargine, the active component of Lantus®; Sanofi-Aventis) whose isoelectric point has been shifted to between 7.0 and 7.4 by modification of the polypeptide sequence of human insulin. A long-acting depot is formed due to precipitation at the pH of the subcutaneous tissue (pH 7.4). Protracted action has also been achieved by covalent modification of insulin by a nonpolar moiety (insulin detemir, the active component of Levimir®; Novo-Nordisk) to augment its hydrophobicity in the subcutaneous depot and to enable binding to serum albumin to delay clearance from the bloodstream. Of historical interest are mixtures of animal insulins (such as porcine and bovine) that exploited their differences in solubility.

The present invention makes novel use of non-axial zinc ions to prolong the duration of action of the insulin analogue formulations provided herein. Prior uses of zinc ions known in the art are as follows. Regular insulin formulations and the corresponding rapid-acting formulations of Humalog® and Novalog® utilize zinc ions to direct and stabilize the assembly of an insulin hexamer. The hexamer consists of three insulin dimers related by a central three-fold axis of symmetry. Each insulin hexamer or insulin analogue hexamer contains two zinc ions located on the three-fold symmetry axis of the hexamer. These “axial zinc ions” are coordinated by the imidazole rings of His^{B10}. In the R₆ hexamer the coordination geometry is thought to be tetrahedral; each zinc ion is thus bound to three symmetry-related His^{B10} residues with the fourth coordination site occupied by a chloride ion. The two axial zinc ions (+4 charge) and two coordinating chloride ions (−2 charge) together add +2 to the total formal charge of the hexamer. There are no non-axial zinc ions in this structure. Single-crystal X-ray diffraction studies of wild-type NPH insulin micro-crystals exhibit two axial zinc ions per hexamer without additional zinc ions. The lattice was orthorhombic with space group P2₁2₁2₁, leading to a pattern of hexamer-hexamer packing inconsistent with the present invention (below).

The majority of insulin products in current use for the treatment of diabetes mellitus contain insulin analogues whose sequence differs from that of natural human insulin. Amino-acid substitutions in the A- and/or B-chains of insulin have widely been investigated for possible favorable effects on the pharmacokinetics of insulin action following subcutaneous injection. Examples known in the art contain substitutions that accelerate or delay the time course of absorption. The former analogues collectively define the “meal-time” insulin analogues because patients with diabetes mellitus may inject such rapid-acting formulations at the time of a meal whereas the delayed absorption of wild-type human insulin or animal insulins (such as porcine insulin or bovine insulin) makes it necessary to inject these formulations 30-45 minutes prior to a meal. The substitutions are designed to destabilize the zinc insulin hexamer by altering the steric or electrostatic complementarity of subunit interfaces and thereby to facilitate the rapid dissociation of the zinc insulin hexamer after subcutaneous administration. Meal-time insulin analogues are formulated as clear solutions at pH 7.4 as zinc-insulin analogue hexamers (Humalog® and Novalog®) or as zinc-free solutions containing monomeric, dimeric, trimeric, tetrameric, and hexameric species in equilibrium (Apidra®; Sanofi-Aventis). Although Humalog® and Novalog® were formulated in phosphate-buffered zinc solutions similar to those long employed in the regular formulations of human insulin and animal insulins, their assembly as zinc insulin hexamers, unlike prior regular formulations known to the art, requires binding of phenol, meta-cresol, or other specific ligands to stabilize the mutant insulin hexamer. It is known in the art that substitution of Pro^{B28} by diverse amino-acid substitutions (excepting Cysteine) destabilizes the zinc insulin hexamer to an extent similar to Asp^{B28} and Lys^{B28}, optionally including substitution of Proline at B29.

Also known in the art are long-acting insulin analogues whose slow absorption over 12-24 hours is intended to provide basal control of blood glucose concentrations. Such analogues, exemplified but not restricted to [Gly^{A21}, Arg^{B31}, Arg^{B32}]-insulin (insulin glargine or Lantus®), may contain amino-acid substitutions and/or extensions of the A- or B-chains designed to shift the isoelectric point of the insulin analogue to between pH 7.0 and 7.4. The analogues are typically formulated as a clear solution containing soluble insulin monomers, dimers, and higher-order oligomers at pH<5 under which conditions zinc-mediated assembly is impaired by protonation of His^{B10}. Prolonged absorption is achieved by aggregation and precipitation of the insulin analogue in the subcutaneous tissue due to a shift in pH to 7.4. The insulin formulation sold as Lantus® contains the active analogue [Gly^{A21}, Arg^{B31}, Arg^{B32}]-insulin (glargine) made 0.6 mM in a solution at pH 4 by addition of aliquots of dilute HCl or NaOH in the presence of inactive components meta-cresol (2.7 mg/ml or 25 mM), glycerol (17 mg/ml or 185 mM), polysorbate-20 (20 μg/ml), and (30 μg zinc ions/ml or 0.52 mM). A U-100 solution of Lantus® contains 0.60 mM [Gly^{A21}, Arg^{B31}, Arg^{B32}]-insulin. Because in wild-type insulin Asn^{A21} is known in the art to undergo acid-catalyzed chemical changes, the purpose of the Gly^{A21} substitution is to avoid such chemical degradation in an acidic solution.

Also known in the art is another type of long-acting insulin analogue is exemplified by insulin detemir (trade name Levemir®) in which residue Thr^{B30} has been deleted and a C₁₄ fatty-acid chain is connected to the side chain of Lys^{B29} (molecular mass 5912.9 Daltons). The fatty acid chain increases the hydrophobicity of the insulin molecule, which is associated with delayed absorption of the subcutaneous depot. The fatty acid chain also mediates binding of the insulin analogue to serum albumin and hence extends its circulating lifetime. Insulin detemir is formulated as soluble zinc-insulin analogue hexamers (14.2 mg/ml or 2.5 mM in insulin monomer units, defined as a U-100 solution) in a clear solution buffered at pH 7.4 by sodium phosphate (0.89 mg/ml of the disodium dihydrate) in the presence of inactive excipients sodium chloride (1.17 mg/ml), meta-cresol (2.06 mg/ml), phenol (1.80 mg/ml mM), mannitol (30 mg/ml), and zinc ions (65.4 Kg/ml or 1.1 mM). The concentration of zinc ions corresponds to a ratio of approximately 2.6 zinc ions per hexamer. The molar activity of insulin detemir is reduced by approximately fourfold relative to wild-type human insulin. The crystal structure of the des-Thr^{B30}/C₁₄-Lys^{B29}-modified insulin analogue in the presence of zinc ions and phenol similar but not identical to that found in its formulation depicts native-like R₆ hexamers with packing of the fatty acid between hexamers in the crystal lattice. The physical state or structure of insulin detemir as is formed in a subcutaneous depot is not known to the art.

There is a need, therefore, for an insulin analogue with a combination of substitutions that can combine to create novel zinc-binding sites at the surface of and between hexamers of the zinc insulin analogue and in so doing to provide formation of a long-acting subcutaneous protein depot.

Insulin belongs to a superfamily of vertebrate insulin-related proteins, including (in addition to insulin itself) insulin-related growth factors I and II (IGF-I and IGF-II), relaxin, and relaxin-related factors. These proteins exhibit homologous α-helical domains and disulfide bridges. IGFs are single-chain polypeptides containing A- and B domains, an intervening connecting (C) domain, and C-terminal D domain; due to proteolytic processing insulin and relaxin-related factors contain two chains (designated A and B). Whereas the six motif-specific cysteines and selected core residues are broadly conserved throughout the vertebrate insulin-related superfamily, other residues are restricted to particular proteins, giving rise to functional specificity. Insulin and IGFs function as ligands for receptor tyrosine kinases (the insulin receptor (IR) and class I IGF receptor (IGF-1R)), whereas relaxin and related factors bind to G-protein coupled receptors (GPCRs). Insulin binds most strongly to IR, weakly to IGF-1R, and is without detectable binding to GPCRs. IGF-I binds most strongly to IGF-1R, weakly to IR, and is without detectable binding to GPCRs. Cross-binding of insulin to IGF-1R can trigger mitogenic signaling pathways, including those associated with proliferation of cancer cells. The long-term safety of insulin replacement therapy in the treatment of diabetes mellitus may be enhanced by use of insulin analogues containing amino-acid substitutions that decrease the extent of such cross-binding. Such amino-acid substitutions would enhance the ratio of affinity of the insulin analogue for IR versus IGF-1R. There is, therefore, a need for a long-acting insulin analogue formulation in which the active component (the component insulin analogue in monomeric form) exhibits decreased intrinsic affinity for IGF-1R and increased ratio of affinity of the insulin analogue for IR versus IGF-1R, in each case relative to the properties of wild-type human insulin.

Insulin glargine binds more strongly than does human insulin to the Type 1 receptor for insulin-like growth factor I (IGF-I). This receptor (IGF-1R) can mediate mitogenic signaling pathways and inhibit apoptosis. The extent of augmented IGF-1R binding and signaling has been estimated to be between a factor of 1.4 and 14 depending on the in vitro or cell-based assay employed. Such augmented IGF-1R binding and signaling are associated with the increased proliferation of human cancer cell lines in culture. The physical state or molecular structure of [Gly^{A21}, Arg^{B31}, Arg^{B32}]-insulin under conditions of formulation or as is formed in the subcutaneous depot is not known in the art.

Concern for the safety of insulin analogues that exhibit increased relative or absolute affinity for IGF-1R was first raised more than ten years ago by the enhanced mitogenicity of Asp^{B10}-insulin in cell culture studies of human cancer cell lines (relative to human insulin) and by an increased incidence of mammary carcinomas in Sprague-Dawley rats being treated by Asp^{B10}-insulin (relative to treatment with human insulin). Asp^{B10}-insulin was accordingly not pursued as a clinical insulin analogue formulation for human use. Recently, analogous concerns have been raised regarding Lantus®, which also exhibits enhanced cross-binding to IGF-1R and increased mitogenicity in human cell culture. A recent retrospective case study of more than 120,000 European patients with diabetes mellitus being treated with Lantus® suggested a dose-dependent increase in the incidence of diverse cancers, including cancers of the breast, prostate, colon, and pancreas. The extent of cancer risk may be increased not only by the elevated level of cross-binding to IGF-1R, but also by the reduced affinity of Lantus® for IR. The receptor-binding selectivity of [Gly^{A21}, Arg^{B31}, Arg^{B32}]-insulin (the ratio of IR association constant to the IGF-1R association constant) is thus anomalously reduced relative to wild-type insulin or other insulin analogues in current clinical use.

Human insulin itself can bind to IGF-1R but with an in vitro affinity for the detergent-solubilized and lectin-purified receptor 333-fold lower than that of its binding to IR. Meal-time insulin analogues such as Humalog® and Novolog® exhibit a similar level of cross-binding to IGF-1R (the cross-binding of insulin lispro to IGF-1R (the active component of Humalog®) has been reported to be slightly increased). Epidemiological studies have revealed an association between endogenous hyperinsulinemia (a compensatory response to insulin resistance in the metabolic syndrome and early stages of type 2 diabetes mellitus) with increased prevalence of cancer, especially colorectal cancer. Treatment of patients with insulin resistance with human insulin or insulin analogues at high doses may also be associated with an increase in cancer risk, which may reflect this baseline level of cross-binding to IGF-1R. For such patients it is possible that even the baseline receptor specificity of human insulin and meal-time insulin analogues may be insufficiently stringent to ensure the safety of long-term treatment with respect to cumulative cancer risk. While not wishing to be constrained by theory, prudence suggests that the receptor-binding selectivities of insulin analogues designed for the treatment of diabetes mellitus should be equal to or greater than the receptor-binding selectivity of wild-type human insulin.

Regulation of blood glucose concentrations by insulin analogues does not require binding to IR at the precise level of human insulin. A decrease in the affinity of an analogue for IR can be compensated in vivo by a delay in its clearance from the bloodstream. Such compensation occurs because clearance of insulin is mediated by its binding to IR. Insulin analogues with threefold reduced affinity for IR can nonetheless exhibit in vivo potencies similar to that of human insulin. Further decreases in affinity can be compensated by an increase in the amount of analogue injected. Examples of insulin analogues with such decreased affinity are insulin glargine (Lantus®) and insulin detemir (Levemir®). Changes in the affinity of insulin analogues for IR usually reflect changes in off rates: reductions in affinity are associated with shortening of the residence time of the hormone on the receptor whereas increases in affinity are associated with prolongation of the residence time. It is not known what in general are the relationships between residence time and metabolic potency or between residence time and mitogenic signaling. Prolonged residence time of Asp^{B10}-insulin on the IR complex has been proposed to underlie, at least in part, its enhanced mitogenicity. While not wishing to be constrained by theory, past experience has taught that insulin analogues with relative in vitro affinities for IR between 20% and 200% relative to human insulin can be effective for the treatment of diabetes mellitus in mammals.

Therefore, there is a need for insulin analogues that exhibit prolonged duration of action with reduced cross-binding to IGF-1R while maintaining at least a portion of the biological activity of the analogue in control of blood glucose concentration. In particular, there is a need for insulin analogues that exhibit delayed absorption from a subcutaneous depot but which, once absorbed into the bloodstream, exhibits decreased IGF-1R affinity while maintaining at least a portion of the biological activity of the analogue in control of blood glucose concentration. There is a further need for insulin analogues that exhibit an increase in isoelectric point toward neutrality without increase in IGF-1R affinity while maintaining at least a portion of the biological activity of the analogue in control of blood glucose concentration.

The biological, physical, and chemical properties of insulin analogues can be altered relative to human insulin due to the presence of amino-acid substitutions in the A-chain and/or B-chain or due to possible extensions of the A-chain and/or B-chain to create a larger molecule. Studies of insulin analogues have indicated that the properties of analogues containing two or more modifications cannot reliably be predicted based on the properties of a set of analogues containing corresponding single modifications. Because an amino-acid substitution or chain extension at one location in the molecule can lead to transmitted changes in the conformation, dynamics, or solvation of the protein, effects of an amino-acid substitution at another location in the molecule can differ from the effects of the same substitution in the absence of the first modification. An example of an unanticipated transmitted effect of a modification is provided by distortions in the crystal structure of Arg^{A0}-insulin, which have been associated with decreased receptor binding. N-terminal extension of the A-chain to include Arg^{A0} thus alters the structural environments of residues A4, A8, and other sites. Amino-acid substitutions or chain extensions that insert one or more basic residues (Arg or Lys) in general result in an upward shift in the isoelectric point toward neutrality; the extent of this shift is influenced by the structure, solvation, and transmitted conformational changes associated with the modification, and so experience has taught that observed pIs are not well predicted by the properties of the isolated amino acids. While not wishing to be restrained by theory, experience has taught that the combined effects of two or more modifications can be unanticipated based on the properties of analogues containing single modifications. It is therefore possible that novel combinations of modifications may together have properties that provide unique advantages for the therapeutic use of insulin analogues in the treatment of diabetes mellitus.

### BRIEF SUMMARY OF THE INVENTION

The present invention pertains to insulin analogue formulations containing multiple Histidine substitutions that can combine to create novel zinc-binding sites at the surface of and between zinc insulin analogue hexamers and in so doing to enable formation of a long-acting subcutaneous protein depot. More particularly, the present invention provides insulin analogues containing paired Histidine substitutions at A4 and A8 with or without a substitution at A21 and provides formulations for their subcutaneous administration to enable prolonged duration of action. Without wishing to condition patentability on any particular theory, side chains at these sites are each believed to project into solvent from the surface of the A-chain on its assembly into an insulin hexamer, thus providing part of a novel zinc-ion-binding site which, in combination with complementary side chain projections from adjoining hexamers, enables zinc-ion-bridged interactions between the adjoining insulin analogue hexamers. As represented in FIG. 1E, the wild-type T₃R^{f}₃ insulin hexamer comprises an upper row (the T₃ trimer; round-cornered rectangle) and lower row (R^{f}₃ trimer; sharp-cornered rectangle), each of which contains an axial zinc ions (gray circles). FIG. 1F provides a schematic representation of the stacking of variant hexamers in the crystal lattice that is believed to take place with the His^{A4} and His^{A8} substitutions of the present formulation. Layers of bridging zinc ions (black circles) are each coordinated by residues His^{A4} and His^{A8} of each T-state protomer (not shown) and His^{A4} side chain from an R^{f}-state protomer above (vertical segment). Also without wishing to condition patentability on any particular theory, this combination of substitutions also enhances the receptor-binding selectivity of the insulin analogues and decreases absolute affinity for IGF-1R.

In another aspect of the present invention, a formulation of a long-acting insulin analogue at about pH 4 is provided, which forms a microcrystalline suspension when its pH is shifted to about 6-7.4. In one particular example, the formulation contains zinc ions at a relative concentration of at least about 4 zinc ions per 6 insulin analogue molecules. The formulation therefore, is capable of subcutaneous injection into an individual, whereupon it forms a subcutaneous depot due to exposure to physiological pH. The formulation may additionally exhibit decreased affinity for the IGF receptor in comparison to wild type insulin of the same species and maintain at least 20% of the affinity of wild-type insulin for the insulin receptor of the same species.

In the native structure of insulin, residues A1-A8 comprise an α-helix. This segment is thought to contribute to the binding of insulin and insulin analogues to both IR and IGF-1R. While not wishing to condition patentability on theory, it is believed that substitutions of solvent-exposed residues Glu^{A4} and Thr^{A8} (not conserved in IGF-I) are well tolerated for binding of insulin analogues to the IR and yet in proximity to the hormone-receptor interface. Substitution of Asn^{A21} by Gly is known in the art to retard the chemical degradation of insulin analogues when formulated under acidic conditions.

It is, therefore, desired to provide insulin analogues that provide zinc-dependent long-acting subcutaneous protein depot and that retain high affinity for the insulin receptor with decreased cross-binding to the Type I IGF receptor. Without wishing to be restrained by theory, it is also desirable to provide insulin analogues in which the two positive charges of bound non-axial zinc ions in an insulin analogue hexamer contribute to a further shift in its assembly-dependent isoelectric point. It is also desirable to provide insulin analogues in which paired Histidine side chains at positions A4 and A8 can contribute to novel interfacial zinc-ion binding sites between insulin analogue hexamers in a crystal lattice. Again without wishing to be restrained by theory, such interfacial zinc ions may retard the disassembly of higher-order contacts between and among hexamers to prolong the duration of action of an insulin analogue.

The A1-A8 α-helix of insulin or of insulin analogues contributes to its isoelectric point (pI) by its combination of charged sites, neutral sites, α-helical dipole moment, and mutual electrostatic interactions. While again not wishing to be constrained by theory, an upward shift in pI toward but not exceeding pH 6.5 would be anticipated by removal of an acidic residue (as occurs on substitution of Glu^{A4} by His). Small changes in pI may be associated with insertion of a Histidine residue at position A4 or A8 depending on the local pKₐ of the substituted Histidine (ordinarily between 6 and 7). While again not wishing to be constrained by theory, an acidic residue is observed in human insulin at position A4. Substitution of Asn^{A21} by Gly, Ala or other neutral side chain would not be expected to cause a significant change in pI; substitution by a basic side chain (Arg or Lys) would be expected to cause a further upward shift in pI; substitution by Asp (as can occur on deamidation of the native Asn^{A21} side chain on storage in acidic solution) would be expected to cause a downward shift in pI. Non-axial zinc ions bound to the surface of the insulin analogue hexamer or bound at interfacial sites between zinc insulin analogue hexamers can also contribute to the total charge of the hexamer or multi-hexamer complex and so affect their solubilities at pH 7.4 as in a subcutaneous depot.

It is therefore also desirable to provide insulin analogues that exhibit the above receptor-binding properties and also exhibit an upward shift in isoelectric point toward but not exceeding neutrality such that, on binding of non-classical zinc ions at the surface of or between insulin analogue hexamers, the combined effects of the amino-acid substitutions and additional bound zinc ions render the complex insoluble at pH 7.4 as in a subcutaneous depot.

It is therefore desirable to provide a soluble formulation of the insulin analogue at acidic pH as a clear solution providing ease of handling, precise adjustment of dose for subcutaneous injection by syringe, and precise metered delivery by pen. It is also desirable to provide a method of crystallization as a basis for a micro-crystalline suspension of the insulin analogue at neutral pH, which may confer added shelf life and product stability at room temperature following initial usage.

In general, a method of treating a patient comprises administering a physiologically effective amount of an insulin analogue or a physiologically acceptable salt thereof to the patient, where the analogue or a physiologically acceptable salt thereof contains an insulin A-chain sequence modified at positions A4 and A8 by a pair of Histidine substitutions with possible additional modification at A21. In one example, the A21 side chain is the native Asn residue. In another example, the A21 side chain is Gly. In another example, the A21 substitution may be Ala, Thr, or Ser.

An insulin analogue may be an analogue of any vertebrate insulin or insulin analogue containing a modified B-chain known in the art to confer altered absorption after subcutaneous injection. In one example, the insulin analogue is a mammalian insulin analogue such as human, murine, rodent, bovine, equine, or canine insulin analogues. In other examples, the insulin analogue is an analogue of sheep, whale, rat, elephant, guinea pig or chinchilla insulin.

Specific insulin analogues include those containing an A-chain sequence as provided by any one of SEQ. ID. NOS. 4-6 or 14 with a B-chain sequence of any one of SEQ. ID. NOS. 7-12. A nucleic acid may encode a polypeptide having one of these sequences. Such a nucleic acid may be part of an expression vector, which may be used to transform a host cell.

### BRIEF DESCRIPTION OF THE SEVERAL VIEWS OF THE DRAWINGS

FIG. 1A is a schematic representation of the sequence of human proinsulin including the A- and B-chains and the connecting region shown with flanking dibasic cleavage sites (filled circles) and C-peptide (open circles).

FIG. 1B provides a structural model of proinsulin, consisting of an insulin-like moiety and disordered connecting peptide (dashed line).

FIG. 1C provides a representation of a proposed pathway of insulin fibrillation via partial unfolding of monomer. The native state is protected by classic self-assembly (far left). Disassembly leads to equilibrium between native- and partially folded monomers (open triangle and trapezoid, respectively). This partial fold may unfold completely as an off-pathway event (open circle) or aggregate to form a nucleus en route to a protofilament (far right).

FIG. 1D is a schematic representation of the sequence of human insulin indicating the position of residue A8 in the A-chain and sites of substitution in the B-chain known in the art to confer rapid absorption after subcutaneous injection.

FIG. 1E is a schematic representation of a wild-type T₃R^{f}₃ insulin hexamer, comprising an upper row (the T₃ trimer; round-cornered rectangle) and lower row (R^{f}₃ trimer; sharp-cornered rectangle), each of which contains an axial zinc ions (gray circles).

FIG. 1F is a schematic representation of the stacking of variant hexamers in a crystal lattice in which layers of three bridging zinc ions (black circles) are each coordinated by residues His^{A4} and His^{A8} of each T-state protomer (round-cornered rectangle) and His^{A4′} side chain (vertical segment) from an R^{f}-state protomer (sharp-cornered rectangle).

FIG. 2a provides the sequence of wild-type insulin and sites of modification in (upper panel) insulin glargine (Lantus®, Sanofi-Aventis) and (lower panel) the present analog. Wild-type A- and B-chain sequences are shown in black and gray; disulfide bridges (A6-A11, A7-B7, and A20-B19) are indicated by black lines. Insulin glargine contains a two-residue extension of the B-chain (Arg^{B31} and Arg^{B32}) and substitution Asn^{A21}→Gly (red in upper panel). Endogenous subcutaneous proteases may slowly remove one or both Arg residues from the extended B-chain of insulin glargine, in part alleviating its augmented mitogenicity. The present analog contains paired (i, i+4) substitutions Glu^{A4}→His and Thr^{A8}→His (in lower panel). Long-acting analog insulin detemir (Levemir®, Novo-Nordisk) operates by attachment of an albumin-binding element (not shown).

FIG. 2b provides a ribbon model of insulin monomer depicting portion of putative zinc-ion binding site formed by His^{A4} and His^{A8} at external surface of A1-A8 α-helix. A- and B-chain ribbons are shown in black and gray, respectively.

FIG. 2c depicts the structure of the wild-type T₃R^{f}₃ insulin hexamer. The two axial zinc ions within the hexamer are aligned at center, coordinated by trimer-related His^{B10} side chains (light gray). The A-chains are shown in black, and B-chains in gray (R^{f}-specific B1-B8 α-helix). The wild-type structure was obtained from the Protein Databank (entry 1TRZ).

FIG. 2d depicts the structure of the variant [His^{A4}, His^{A8}] T₃R^{f}₃ insulin hexamer. The two axial zinc ions within the hexamer are aligned at center, coordinated by trimer-related His^{B10} side chains (light gray). The variant hexamer contains three non-classical zinc ions at the T₃ trimer surface (peripheral spheres). Shown in gray are the side chains of His^{A4}, His^{A8}, and third His^{A4′} from adjoining hexamer. In each case the A-chains are shown in black, and B-chains in gray (R^{f}-specific B1-B8 α-helix).

FIG. 2e illustrates 2Fₒ-F_{c} electron-density map (stereo pair contoured at 1 s) showing novel zinc-ion binding site formed by His^{A4} and His^{A8} in T-state protomer. Distorted tetrahedral coordination is completed by residue A4′, belong to an R^{f}-state protomer in adjoining hexamer.

FIG. 3A depicts the wild-type hexamer-hexamer packing. (Left) In each hexamer the upper trimer has T₃ conformation, and lower trimer R^{f}₃. Axial zinc ions (larger spheres), and interfacial water molecules (smaller spheres) near residues A4 and A8 are shown. A-chains are shown in gray, and B-chains black. T- and R protomers differ in B1-B9 secondary structure, extended (T) or helical (R); residues B1 and B2 are disordered in the “frayed” R^{f} state. (Right) Expansion of boxed region at left. Larger sphere toward bottom is axial zinc ion of T₃ trimer in bottom hexamer. Arrows indicate R^{f}-state residues Glu^{A4′} in upper R^{f}₃ trimer.

FIG. 3B depicts the zinc-mediated hexamer-hexamer packing of [His^{A4}, His^{A8}]-insulin: upper trimer has T₃ conformation, and lower trimer R. Axial zinc ions and A4-A8-A4′ coordinated zinc ions are shown. A-chains are shown in gray, and B-chains in black. (Right) Expansion of boxed region. Three novel zinc ions are observed at hexamer-hexamer interface. Arrows indicate R^{f}-state side chain His^{A4′} (from bottom trimer of top hexamer), which complete the interfacial zinc-binding sites.

FIG. 3C provides CPK models showing T and R^{f} faces of [His^{A4}, His^{A8}]-insulin hexamer (left and right). View shown in rotated by 90° relative to panel FIG. 3b. The three non-classical zinc ions are shown bound to side chains of His^{A4} and His^{A8}. White crosses indicate position of chloride ions; the scheme is otherwise as in FIG. 3B.

FIG. 3D provides a stereo pair showing non-classical zinc ion (large dark gray sphere), chloride ion (overlapping light gray sphere), and three bound water molecules (smaller spheres) in relation to His^{A4′} in R^{f} protomer and His^{A4}-His^{A8} in T protomer. The bound water molecules participate in a hydrogen-bond network within R^{f} involving the side-chain carboxylate of Glu^{B4′}, para-OH of Tyr^{B26′}, and carbonyl oxygen of Pro^{B28′} (labeled).

FIG. 3E depicts the results of competitive displacement assays probing high-affinity binding of insulin or insulin analogs to IR (left-hand three curves; solid lines) and low-affinity cross-binding to IGF-1R (right-hand three curves; dotted lines). In each group results for wild-type insulin (x), insulin glargine (▪), and His^{A4}, His^{A8}-insulin (▾) are shown. The enhanced receptor-binding selectivity of His^{A4}, His^{A8}-insulin results from leftward shift of its IR-binding titration and rightward shift of its IGF-1R-binding titration. Relative affinities and dissociation constants are provided in Tables 2 and 3. Assays were performed in the absence of zinc ions.

FIG. 3F provides results of in vivo assays. Steptozotocin-induced diabetic male rats were injected subcutaneously with either wild-type insulin (x), insulin glargine (▪), His^{A4}, His^{A8}-insulin (▾), or buffer control (Lilly diluent; ). Doses at time 0 were 3.44 nmoles wild-type insulin (20 mg in 100-μ1 injection volume), 12 nmoles insulin glargine (corresponding to 2.0 U Lantus®), 13.7 nmoles [His^{A4}, His^{A8}]-insulin, and 100-μl protein-free buffer (Lilly diluent). Blood glucose concentration was measured from the tip of the tail at indicated times. Each analog was tested in 5 rats (mean±SEM); experiment was repeated 2 times with similar results. Rats were fed 6-8 h following injections.

FIG. 4 provides a graph showing the blood glucose levels (in mg/dL) over time in steptozotocin-induced diabetic male rats after injection with insulin diluent as a control (circles), insulin glargine (Lantus®, squares), lispro insulin (Humalog®, “X”) or the insulin analogue containing His substitutions at positions A4 and A8 and the lispro substitutions of Humalog (A4A8-lispro+Zn, inverted triangles), as otherwise provided above with regard to FIG. 3f.

FIG. 5 is a schematic representation of the use of Histidine substitutions to permit zinc-mediated associations of proteins to create long-acting depots of protein in question.

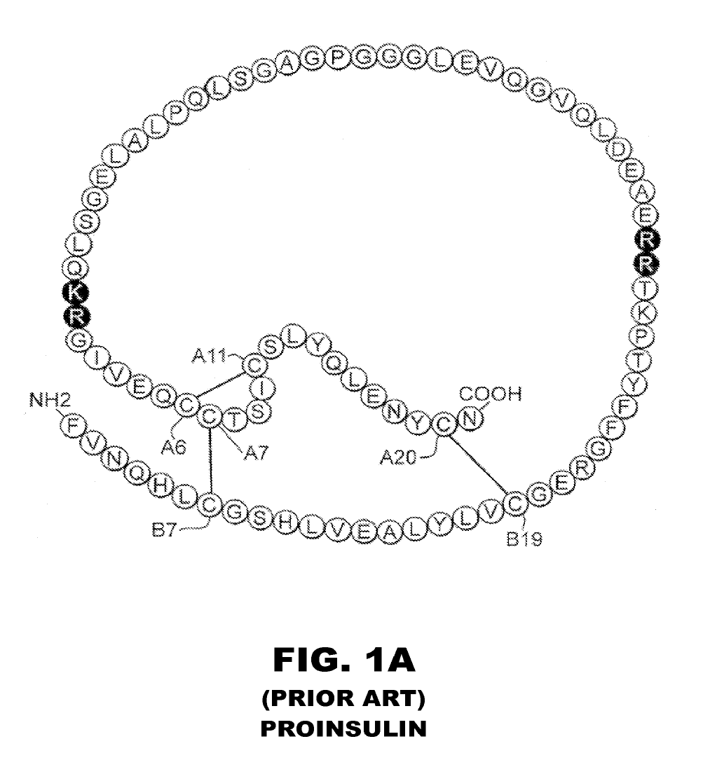

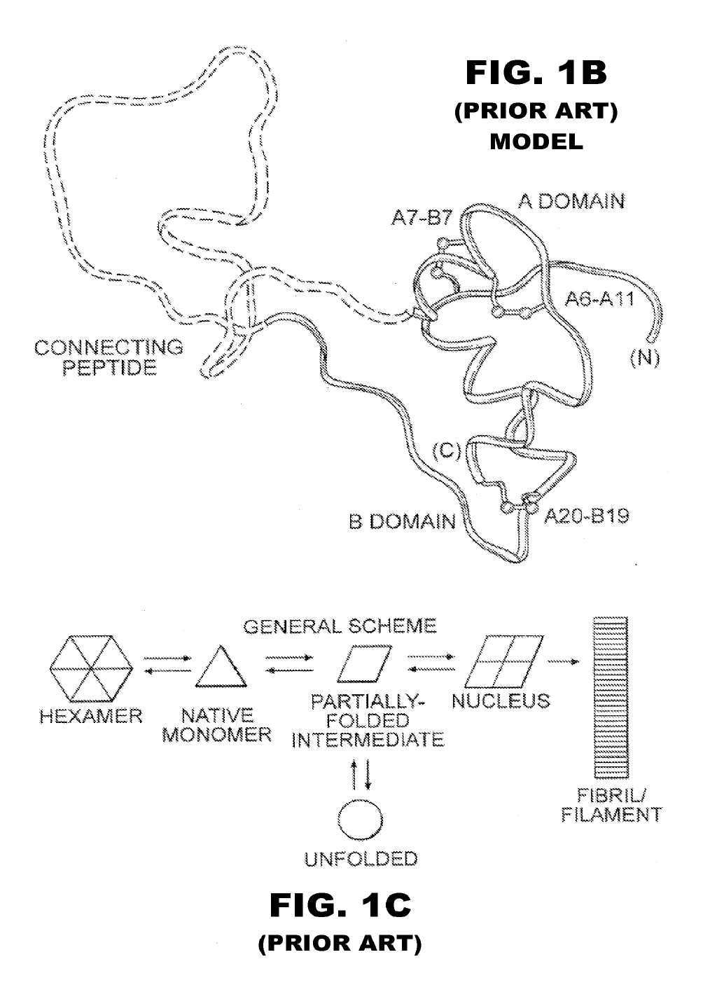

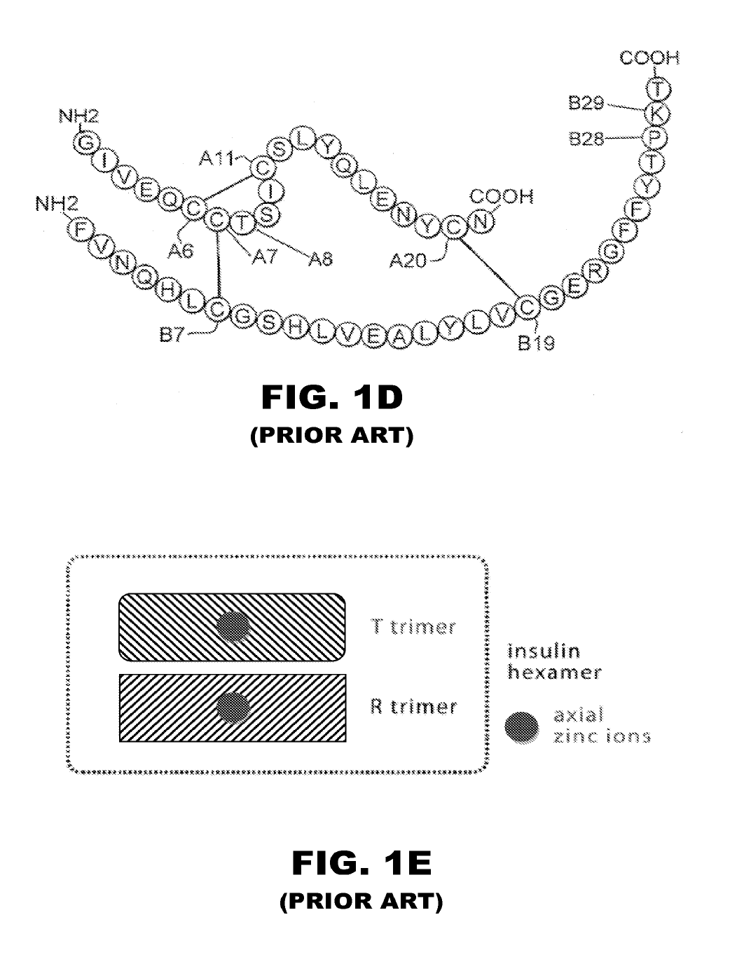

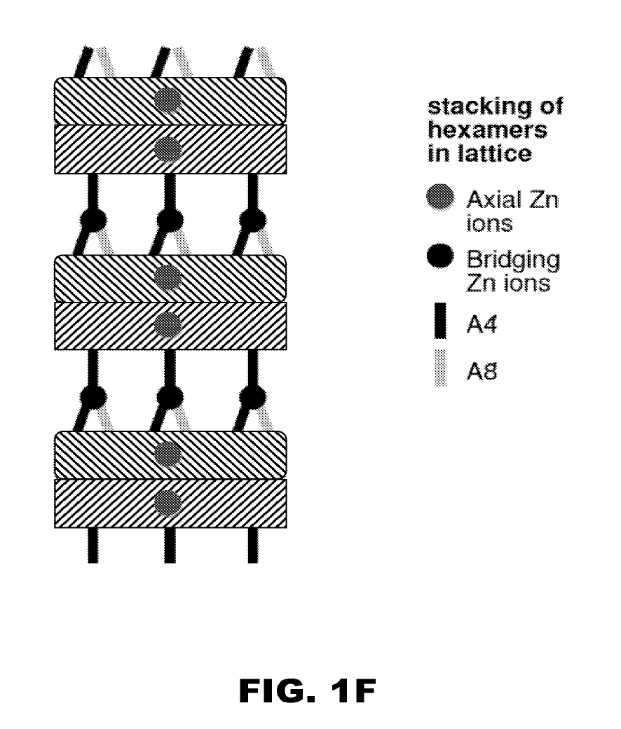

### DETAILED DESCRIPTION OF THE INVENTION

The present invention is directed toward the novel use of non-axial interfacial zinc ions between insulin hexamers to prolong the duration of action of an insulin analogue formulation. The present invention provides a new system for creating a prolonged subcutaneous depot. It makes use of novel non-axial zinc ions to bind at the surface of and between insulin analogue hexamers and to prolong the time it takes for depots of these analogues to release monomeric insulin analogue to the bloodstream. The invention also provides for concomitant decrease in the absolute and relative binding of insulin analogues to the Type 1 IGF receptor. This combination of properties will enhance the efficacy and safety of treatment of diabetes, particularly with respect to the risk of cancer. To that end, the present invention provides insulin analogues that contain paired Histidine amino-acid substitutions at positions A4 and A8 together with zinc-containing formulations, either as a clear solution at pH 4 or as a micro-crystalline suspension around neutral pH. The paired A4-A8 substitutions may be combined with a substitution at position A21, such as Gly, Ala, Ser, or Thr.

The insulin analogues of the present invention may also contain other modifications. As used in this specification and the claims, various substitutions in analogues of insulin may be noted by the convention that indicates the amino acid being substituted, followed by the position of the amino acid, optionally in superscript. The position of the amino acid in question includes the A- or B-chain of insulin where the substitution is located. For example, an insulin analogue of the present invention may also contain a substitution of Aspartic acid (Asp or D) or Lysine (Lys or K) for Proline (Pro or P) at amino acid 28 of the B-chain (B28), or a substitution of Pro for Lys at amino acid 29 of the B-chain (B29) or a combination thereof. These substitutions may also be denoted as Asp^{B28}, Lys^{B28}, and Pro^{B29}, respectively. Similarly, an insulin of the present invention may contain an addition of Arginine (Arg or R), Histidine (His or H), or Lysine (Lys or K) at amino acid A0 of the A-chain (i.e., N-terminal to Gly^{A1}). These additions may be denoted Arg^{A0}, His^{A0}, or Lys^{A0}, respectively. Further, the present substitutions may be combined with introduction of the substitution Phe^{B1}→His. Unless noted otherwise or wherever obvious from the context, the amino acids noted herein should be considered to be L-amino acids.

As used herein, a “metal-staple” or “metal-zipper” is a metal-binding site formed when two or more amino-acid side chains from two or more molecules or molecular complexes associate with the metal in question. For example, one side chain from a first molecule may combine with two side chains from a second molecule to create a zinc-binding site. It is well known in the art that insulin trimers are thus “zinc-stapled” together by an axial zinc ion. However, it has not been previously known that zinc-bonding sites might be introduced to cause hexamers of certain insulin analogs to form zinc-staples between hexamers.

The invention provides insulin analogues that form novel “zinc-stapled” insulin hexamer-complexes and exhibit reduced affinity for IGF-1R while retaining at least a portion of their affinity for IR and hence biological activity. The invention also provides formulations of these analogues with high relative concentrations of zinc, which form “zinc-stapled” hexamer complexes and, at even higher concentrations of zinc, Lente-like crystals of these hexamer-complexes. In some embodiments, the insulin analogues contain at least 4, at least 5, at least 6, at least 7, or at least 8 zinc ions per hexamer of the insulin analogue.

A method for treating a patient comprises administering an insulin analogue to the patient. In one example, the insulin analogue is an insulin analogue containing modifications in the A-chain that concomitantly cause an upward shift in isoelectric point (pI) toward neutrality, permit the assembly of zinc-stapled insulin hexamers. In another example, the modifications also reduce the affinity of the zinc-free monomer for IGF-1R. In another example, the insulin analogue also contains a substitution at position A21 that protects the insulin analogue from chemical degradation when formulated under acidic conditions. The insulin analogue is administered by subcutaneous injection using a syringe, metered pen, or other suitable device.

It is also envisioned that it would be possible to apply the introduction of paired Histidine substitutions at positions A4 and A8 to analogues formulated with a sufficient concentration of zinc ions at acidic pH would render the analogues insoluble at pH 7.4 by two concurrent mechanisms: a shift toward higher isoelectric point (ca. 6.5-6.6) due primarily to removal of the negative charge of Glu^{A4} and a further shift of the net isoelectric point of the zinc insulin hexamer due to binding of non-axial zinc ions in addition to classical axial zinc ions. The same substitutions at A4 and A8 introduced for the purposes of decreasing the solubility of the insulin analog in the subcutaneous depot will also reduce a possible cancer risk proposed to be associated with cross-binding of insulin and insulin analogues to the Type 1 IGF receptor.

It is further envisioned that it would be possible to apply the introduction of combined substitutions at positions A4 and A8, with or without substitutions at A21, in other classes of formulations of insulin analogues (such as but not restricted to regular, NPH, semi-lente and lente, including mixtures of such types) for one or more of the purposes of decreasing cross-binding of such analogues to the Type 1 IGF receptor. For the purpose of a regular soluble formulation at pH 7.4 the paired Histidine substitutions must be combined with substitutions elsewhere in the A- or B-chains that remove one or more positive charges or add one or more negative charges, thereby lowering the pI sufficiently to enable solubility like that of human insulin at pH 7.4 in the presence of excipients known in the art, including but not limited to zinc chloride, phenol, meta-cresol, glycerol, sodium phosphate buffer, and water for injection. Examples of substitutions that would lower the pI when combined with the paired hisidine substitutions at A4 and A8 include, but are not limited to, Glu^{A14}, Asp^{A21}, Glu^{A21}, Asp^{B9}, Glu^{B9}, Asp^{B10}, Glu^{B10}, Ala^{B22}, Ser^{B22}, Asp^{B28}, Asp^{B}28-Pro^{B29}, Asp^{B28}-Ala^{B29}, Ala^{B29}, and Pro^{B29}; or combinations thereof.

It has been discovered that paired Histidine substitutions at positions A4 and A8 can reduce cross-binding by an insulin analogue to the Type I IGF receptor and effect an upward shift in pI toward neutrality while maintaining native affinity for the insulin receptor.

It has also been discovered that [His^{A4}, His^{A8}]-insulin is highly soluble when made zinc-free at pH 7.4 but addition of 4-6 zinc ions per 6 insulin analogue molecules results in precipitation of a zinc-protein complex. This complex is insoluble or sparingly soluble in the pH 7.0-8.4, but is soluble at about pH 4. While not wishing to be constrained by theory, it is likely that this pH-dependent insolubility is due to the in vitro isoelectric precipitation of a zinc protein complex containing both axial and non-axial bound zinc ions.

It has also been discovered that [His^{A4}, His^{A8}]-insulin, when formulated in an unbuffered solution at pH 4.0 and containing a molar ratio of 5.2 zinc ions per 6 insulin analogue molecules and following subcutaneous injection in a male Lewis rat rendered diabetic by streptozotocin, will direct prolonged control of blood glucose concentrations to a duration and extent similar to the pharmacologic action of Lantus®. While not wishing to be constrained by theory, it is likely that this prolonged action is due to subcutaneous isoelectric precipitation of a zinc protein complex containing both axial and non-axial bound zinc ions.

It has also been discovered that crystals of [His^{A4}, His^{A8}]-insulin may readily be grown as zinc insulin analogue hexamers containing two axial zinc ions per hexamer and three non-axial zinc ions, bound between successive hexamers in the R3 crystal lattice; the latter interfacial zinc ions exhibit tetrahedral coordination by His^{A4} and His^{A8} in one hexamer, His^{A4′} in the adjoining hexamer, and a bound chloride ion. The three bound zinc- and chloride ions add formal changes of +6 and −3 to the hexamer, respectively, with net formal change of +3. These additional changes extend the formal change of +6 achieved by the substitution of Glu^{A4} by His. While not wishing to be constrained by theory, it is likely that the presence of three non-axial zinc ions per hexamer leads to the above pH-dependent insolubility and presumed subcutaneous isoelectric precipitation of a zinc protein complex.

In general, a vertebrate insulin analogue or a physiologically acceptable salt thereof, comprises an insulin analogue containing an insulin A-chain and an insulin B-chain. An insulin analogue of the present invention may also contain other modifications, such as substitutions of a basic amino-acid extensions of the B-chain at residues B1 and/or B31. In one example, the vertebrate insulin analogue is a mammalian insulin analogue, such as a human, porcine, bovine, feline, canine or equine insulin analogue. An insulin analogue of the present invention may also contain other modifications, such as a tether between the C-terminus of the B-chain and the N-terminus of the A-chain as described more fully in co-pending U.S. patent application Ser. No. 12/419,169, the disclosure of which is incorporated by reference herein.

A pharmaceutical composition may comprise such insulin analogues and to achieve extended duration of action must include zinc ions or another divalent metal ions able to direct protein assembly and interfacial stapling of hexamers Zinc ions may be included in such a composition at a level of a molar ratio of between 4.0 and 7.0, or between 5.0 and 6.0 per hexamer of the insulin analogue Zinc ions may be included at a higher molar ratio as well in order to create hexamer-complexes with even slower absorption; at such higher molar ratios zinc ions would occupy weak zinc-binding sites in addition to the interfacial [His^{A4}, His^{A8}]-related zinc-stapled binding sites. In such a formulation, the concentration of the insulin analogue would typically be between about 0.1 and about 3 mM.

Excipients may include glycerol, Glycine, other buffers and salts, and anti-microbial preservatives such as phenol and meta-cresol; the latter preservatives are known to enhance the stability of the insulin hexamer. Such a pharmaceutical composition may be used to treat a patient having diabetes mellitus or other medical condition by administering a physiologically effective amount of the composition to the patient.

A nucleic acid comprising a sequence that encodes a polypeptide encoding an insulin analogue containing a sequence encoding an A-chain with a combination of Histidine substitutions at A4 and A8, with or without an additional substitution at A21. The particular sequence may depend on the preferred codon usage of a species in which the nucleic acid sequence will be introduced. The nucleic acid may also encode other modifications of wild-type insulin. The nucleic acid sequence may encode a modified A- or B-chain sequence containing an unrelated substitution or extension elsewhere in the polypeptide or modified proinsulin analogues. The nucleic acid may also be a portion of an expression vector, and that vector may be inserted into a host cell such as a prokaryotic host cell like an E. coli cell line, or a eukaryotic cell line such as S. cereviciae or Pischia pastoris strain or cell line.

It is further envisioned that unrelated substitutions or chain extensions can be combined within the analogues of the present invention to modify its isoelectric point, either further upward as by substitutions at position B13 or chain extensions by Arg or Lys at positions A0, A22, B0, or B31; or downward as by substitutions that insert a negative charge or remove a positive charge. For example, the substitutions might be combined with AlaB31-HisB32-HisB33-ArgB34, HisB31-HisB32, HisB31-HisB32-ArgB33, or AlaB31-HisB32-HisB33. In these latter cases, additional intrahexamer zinc binding complements the interhexamer zinc binding of the present invention to raise the zinc to hexamer ratio and further-stabilize the hexamer-complexes.

The substitutions of the present invention may also be combined with B-chain modifications that augment IGF-1R cross-binding to mitigate this unfavorable property; examples include extension of the B-chain by one or two basic residues (such as Arg^{B31}, Lys^{B31}, Arg^{B31}-Arg^{B32}, Arg^{B31}-Lys^{B32}, Lys^{B31}-Arg^{B32}, and Lys^{B31}-Lys^{B32}) or substitution of His^{B10} by Asp or Glu. An example is provided by (but not restricted to) insulin glargine (Lantus®), which is formulated at pH 4 but which undergoes aggregation in a subcutaneous depot at physiological pH.

It is further envisioned that the paired Histidine substitutions of the present invention may also be utilized in combination with any of the changes present in existing insulin analogues or modified insulins, or with various pharmaceutical formulations, such as regular insulin, NPH insulin, lente insulin or ultralente insulin. The insulin analogues of the present invention may also contain substitutions present in analogues of human insulin that, while not clinically used, are still useful experimentally, such as DKP-insulin, which contains the substitutions Asp^{B10}, Lys^{B28} and Pro^{B29} or the Asp^{B9} substitution. The present invention is not, however, limited to human insulin and its analogues. It is also envisioned that these substitutions may also be made in animal insulins such as porcine, bovine, equine, and canine insulins, by way of non-limiting examples. Furthermore, in view of the similarity between human and animal insulins, and use in the past of animal insulins in human diabetic patients, it is also envisioned that other minor modifications in the sequence of insulin may be introduced, especially those substitutions considered “conservative” substitutions. For example, additional substitutions of amino acids may be made within groups of amino acids with similar side chains, without departing from the present invention. These include the neutral hydrophobic amino acids: Alanine (Ala or A), Valine (Val or V), Leucine (Leu or L), Isoleucine (Ile or I), Proline (Pro or P), Tryptophan (Trp or W), Phenylalanine (Phe or F) and Methionine (Met or M). Likewise, the neutral polar amino acids may be substituted for each other within their group of Glycine (Gly or G), Serine (Ser or S), Threonine (Thr or T), Tyrosine (Tyr or Y), Cysteine (Cys or C), Glutamine (Glu or Q), and Asparagine (Asn or N). Basic amino acids are considered to include Lysine (Lys or K), Arginine (Arg or R) and Histidine (His or H). Acidic amino acids are Aspartic acid (Asp or D) and Glutamic acid (Glu or E).

The amino acid sequence of human proinsulin is provided, for comparative purposes, as SEQ. ID. NO. 1. The amino acid sequence of the A-chain of human insulin is provided as SEQ. ID. NO. 2. The amino acid sequence of the B-chain of human insulin is provided, for comparative purposes, as SEQ. ID. NO. 3.

(proinsulin)
SEQ. ID. NO. 1
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn
(A-chain)
SEQ. ID. NO. 2
Gly-Ile-Val-Glu-Gln-Cys-Cys-Thr-Ser-Ile-Cys-Ser-
Leu-Tyr-Gln-Leu-Glu-Asn-Tyr-Cys-Asn
(B-chain)
SEQ. ID. NO. 3
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr

It is envisioned that the insulin analogues of the present invention have affinities for the insulin receptor similar to that of natural insulin but exhibit decreased affinity for the Type 1 IGF receptor. Insulin or insulin analogue activity may be determined by receptor binding assays as described in more detail herein below. Relative activity may be defined in terms of hormone-receptor dissociation constants (K_{d}), as obtained by curve fitting of competitive displacement assays, or in terms of ED₅₀ values, the concentration of unlabelled insulin or insulin analogue required to displace 50 percent of specifically bound labeled human insulin such as a radioactively-labeled human insulin (such as ¹²⁵I-labeled insulin) or radioactively-labeled high-affinity insulin analog. Alternatively, activity may be expressed simply as a percentage of normal insulin. Affinity for the insulin-like growth factor receptor may also be determined in the same way with displacement from IGF-1R being measured. In particular, it is desirable for an insulin analogue to have an activity that is 20-200 percent of insulin, such as 25, 50, 110, 120, 130, 140, 150, or 200 percent of normal insulin or more, while having an affinity for IGF-1R that is less than or equal to 50 percent of normal insulin, such as 10, 20, 30 or 50 percent of normal insulin or less. An insulin analogue can still be useful in the treatment of diabetes even if the in vitro receptor-binding activity is as low as 20% due to slower clearance.

Synthesis of Insulin Analogs.

Chain combination was effected by interaction of the S-sulfonated derivative of the A chain (41 mg) and B-chain analog (21 mg) in 0.1 M Glycine buffer (pH 10.6, 10 ml) in the presence of dithiothreitol (7 mg). CM-52 cellulose chromatography of each combination mixture enabled partial isolation of the hydrochloride form of the protein contaminated by free B-chain. Final purification was accomplished by reverse-phase HPLC. The predicted molecular mass of [His^{A4}, His^{A8}]-insulin was verified by MALDI mass spectrometry. The final yield (6.1 mg) was similar to those obtained in a control synthesis of wild-type insulin. The corresponding yield of [His^{A4}, His^{A8}]-DKP-insulin was 8.8 mg.

Isoelectric Focusing Electrophoresis.

The pI values of insulin and insulin analogs in their native states were measured by IEF gel electrophoresis using pre-cast pH 3-10 IEF gels, (125×125 mm, 300 μm, SERVALYT® Precotes® from SERVA Electrophoresis GmbH, Heidelberg; obtained from Crescent Chemical Co. Hauppauge, N.Y.). The Precotes® were set up in a horizontal IEF apparatus, Multiphor II (Pharmacia Biotech) according to the manufacturer's protocol. The unit was pre-cooled to 4° C. using a circulating water bath (Brinkman), before placing the PRECOTE IEF gel on electrophoresis bed coated with light mineral oil for efficient heat exchange. The gels were connected to electrodes using filter paper wicks wetted with Anode Fluid pH 3 and Cathode Fluid pH 10 (both from SERVA) at the two ends of the gel. Prior to loading the samples, the gel was pre-focused at an initial voltage setting of 200 volts and a final setting of 500 volts for 30 min using a high voltage power supply (LKB model 2197). After loading the samples and the IEF standards (5-10 μL, at a loading protein concentration of 5-10 ng), isoelectric focusing was performed at 500-2000 volts for 2 hrs or until the final voltage of 2000 volts was reached, after which focusing was continued for an additional 15 min. After IEF, the gel was fixed with 200 ml of 20% trichloroacetic acid for 20 min, rinsed for 1 min with 200 ml of deionized water and stained with Serva Violet 17 solution and destained with 86% phosphoric acid according to the SERVA manual protocols. The IEF standard proteins (from SERVA) used are as follows, with their respective pI's in parentheses: horse heart cytochrome C, (10.7), bovine pancreas ribonuclease A (9.5), lens culinaris Lectin (8.3, 8.0, 7.8), horse muscle Myoglobin (7.4, 6.9), bovine erythrocytes Carbonic anhydrase (6.0), bovine milk β-lactoglobin (5.3, 5.2), soybean trypsin inhibitor (4.5), Aspergillus niger glucose oxidase (4.2), Aspergillus niger amyloglucosidase (3.5). The pI's of the protein samples were determined by comparison to a linear regression plot of migration distance versus pH gradient of the IEF standards.

Plasmids of Receptor Expression.

For expression of epitope-tagged IR and IGF-1R, the mammalian expression vector pcDNA3.1Zeo+ was obtained from InVitrogen and was modified for C-terminal epitope tagging by subcloning an in-frame oligonucleotide cassette encoding in-frame triple repeats of the FLAG M2 epitope (Asp-Tyr-Lys-Asp-Asp-Asp-Asp-Lys) between the BamHI and XbaI restriction sites. Respective cDNAs encoding IGF-1R and the B-isoform of IR were as previously described (Whittaker, J. et al. Proc. Natl. Acad. Sci. USA Vol 84, pp. 5237-5241 (1987)). They were modified for subcloning into the modified expression vector by introduction of a BamHI site encoding an in-frame C-terminal Gly-Ser linker at their 3′ ends just prior to the stop codon by site-directed mutagenesis.

Expression of Receptor cDNAs.

DNA for transfection was prepared as previously described. The receptor cDNAs were expressed transiently in PEAK rapid cells using polyethyleneimine. Cells were harvested three days post-transfection when receptor expression was maximal. Lysis was accomplished in a buffer consisting of 0.15 M NaCl and 0.1M Tris-Cl (pH 8.0), containing 1% (v/v) Triton X-100 and a protease inhibitor cocktail (Roche). Lysates were stored at −80° C. until required for assay.

Receptor Binding Assays.

Respective IGF-1R and IR-B binding assays were performed by a modification of the microtiter plate antibody-capture assay that Whittaker and colleagues have described previously. Microtiter strip plates (Nunc Maxisorb) were incubated overnight at 4° C. with anti-FLAG IgG (100 μl/well of a 40 μg/ml solution in phosphate-buffered saline). Washing and blocking were performed as previously described. Detergent lysates of 293 PEAK cells transiently transfected with cDNAs encoding full-length IR-B or IGF-1R with C-terminal FLAG-tags were partially purified by wheat germ agglutinin (WGA) chromatography to deplete lysates of receptor pre-cursors. Wheat-germ eluates were then incubated in the antibody-coated plates for 1 hour at room temperature to immobilize receptors. After extensive washing to remove unbound proteins, competitive binding assays with labeled insulin tracer (¹²⁵I-[Tyr^{A14}]-insulin) or labeled IGF-I tracer (¹²⁵I-Tyr³¹-IGF-I) and unlabeled insulin analogs were carried out as described. All insulin analogs were assayed with either insulin or IGF-I receptor as control ligands in the same set of assays. Binding data from homologous competition assays were analyzed by non-linear regression analysis using a 2-site sequential model to obtain dissociation constants for insulin and IGF-I. Binding data for heterologous competition experiments were analyzed by the method of Wang; this method uses an exact mathematical expression to describe the competitive binding of two different ligands to a receptor.

Representative binding studies of insulin analogues known in the art are summarized in Table 1. Because the affinity of insulin for IR (isoform B) is similar to the affinity of IGF-I for IGF-1R (in each case with K_{d} ca. 0.04 nM), the ratio of respective percent affinities for IR and IGF-1R (columns 2 and 3), as given in column 4, provides an estimate of the absolute specificity of the insulin analogue. Normalization relative to the specificity of human insulin (row 1) provides an estimate of relative specificity. Relative specificities greater than 1 (less than 1) indicate enhanced (decreased) stringency of receptor binding. In this assay Asp^{B10}-insulin exhibits increased affinity for IGF-1R, but because affinity for IR is more markedly increased, the relative specificity is greater than 1. Insulin glargine (Lantus®), which contains substitution Asn^{A21}→Gly and a two-residue extension of the B-chain (Arg^{B31} and Arg^{B32}), exhibits increased absolute affinity for IGF-1R, decreased absolute affinity for IR, and decreased relative stringency of receptor binding. The insulin analogues of the present invention exhibit the opposite property: decreased absolute affinity for IGF-1R and increased relative stringency of receptor binding.

TABLE 1
Receptor-Binding Properties of Control Insulin Analogues^{a}

| | relative affinities^{b} | | receptor selectivity | |
|---|---|---|---|---|
| analogue | IR | IGF-1R | ratio | relative |
| human insulin | 100 | 0.30 ± 0.02 | 333 ± 36 | 1 |
| (ins) | | | | |
| human IGF-I | ND^{c} | 100 | ND | ND |
| lispro-insulin | 92 ± 4 | .27 ± 0.03 | 341 ± 52 | 1.0 ± 0.3 |
| Asp^{B10}-insulin | 336 ± 34 | 0.67 ± 0.07 | 501 ± 100 | 1.5 ± 0.4 |
| DKP-insulin | 236 ± 35 | 1.60 ± 0.24 | 148 ± 49 | 0.4 ± 0.2 |
| Lantus^{e} | 43 ± 7 | 0.92 ± 0.12 | 47 ± 9 | 0.14 ± 0.02 |

^{a}Errors derived from standard errors of the mean.
^{b}The relative affinity of wild-type insulin for IR (column 2) is defined at 100 percent; the relative affinity of IGF-I for IGF-1R (column 3) is also defined as 100 percent. Respective absolute dissociation constants are similar.

A-chain analogues of insulin containing novel combinations of A-chain amino-acid substitutions were made by total chemical synthesis of the variant A-chain. Wild-type B-chains were obtained from commercial formulations of human insulin by oxidative sulfitolysis; the DKP B-chain was likewise prepared by total chemical synthesis. The insulin analogues were in each case obtained by insulin chain combination followed by chromatographic purification. In each case the predicted molecular mass was verified by mass spectrometry.

Insulin analogues were synthesized containing the paired Histidine substitutions at positions A4 and A8, with or without substitution of Asn^{A21} by Gly, are shown generally as SEQ. ID. NO. 4, in the context of a wild-type human B-chain (SEQ. ID. NO. 3). Comparison of the properties of these analogues with human insulin indicates the general effects of A1, A8 substitutions to reduce the affinity of the analogues for IGF-1R and increase the ratio of affinity for IR versus IGF-1R (Table 2).

(paired Histidine substitutions at A4 and A8)
SEQ. ID. NO. 4
Gly-Ile-Val-His-Gln-Cys-Cys-His-Ser-Ile-Cys-Ser-
Leu-Tyr-Gln-Leu-Glu-Asn-Tyr-Cys-Xaa
Xaa = Asn, Gly, Ala, Ser, Thr

TABLE 2
Receptor-Binding Properties of Insulin Analogues^{a}

| | IR-B | | IGF-1R | |
|---|---|---|---|---|
| | K_{d} (nM) | SEM | K_{d} (nM) | SEM |
| insulin | 0.060 | 0.009 | 12.2 | 1.8 |
| Lantus | 0.110 | 0.016 | 3.1 | 0.44 |
| [His^{A4,A8}]-HI | 0.045 | 0.007 | 71.2 | 14.7 |
| [His^{A4,A8}-Gly^{A21}]-HI | 0.091 | 0.012 | 133.3 | 33 |

^{a}IR-B designates isoform B of the human insulin receptor; IGF-1R designates the human Type 1 IGF receptor; SEM, standard error of the mean.

Insulin analogues having the A-chain polypeptide sequences of SEQ. ID. NOS. 5 or 6 and 20-21 were likewise prepared either with wild type insulin B-chain (SEQ. ID. NO. 3) or an insulin analogue such as insulin glargine. An upward shift in isoelectric points to a value of 6.6 in the absence of zinc ions (from a baseline value of 5.6 found for zinc-free human insulin) was verified by isoelectric focusing gel electrophoresis. To this end, studies employed pre-cast pH 3-10 IEF gels, (125×125 mm, 300 μm, SERVALYT® Precotes® from SERVA Electrophoresis GmbH, Heidelberg; obtained from Crescent Chemical Co. Hauppauge, N.Y.). The Precotes® were set up in a horizontal IEF apparatus, Multiphor II (Pharmacia Biotech) according to the manufacturer's protocol. The unit was pre-cooled to 4° C. using a circulating water bath (Brinkman), before placing the PRECOTE IEF gel on electrophoresis bed coated with light mineral oil for efficient heat exchange. The gels were connected to electrodes using filter paper wicks wetted with Anode Fluid pH 3 and Cathode Fluid pH 10 (both from SERVA) at the two ends of the gel. Prior to loading the samples, the gel was pre-focused at an initial voltage setting of 200 volts and a final setting of 500 volts for 30 min using a high voltage power supply (LKB model 2197). After loading the samples and the IEF standards (5-10 μL, at a loading protein concentration of 5-10 μg), isoelectric focusing was performed at 500-2000 volts for 2 hrs or until the final voltage of 2000 volts was reached, after which focusing was continued for an additional 15 min. After IEF, the gel was fixed with 200 ml of 20% trichloroacetic acid for 20 min, rinsed for 1 min with 200 ml of deionized water and stained with Serva Violet 17 solution and destained with 86% phosphoric acid according to the SERVA manual protocols. The IEF standard proteins (from SERVA) used are as follows, with their respective pI's in parentheses: horse heart cytochrome C, (10.7), bovine pancreas ribonuclease A (9.5), lens culinaris Lectin (8.3, 8.0, 7.8), horse muscle Myoglobin (7.4, 6.9), bovine erythrocytes Carbonic anhydrase (6.0), bovine milk β-lactoglobin (5.3, 5.2), soybean trypsin inhibitor (4.5), Aspergillus niger glucose oxidase (4.2), Aspergillus niger amyloglucosidase (3.5). The pI's of human insulin or the insulin analogues of the present invention were determined by comparison to a linear regression plot of migration distance versus pH gradient of the IEF standards.

(His^{A4}, His^{A8} substitutions)
SEQ. ID. NO. 5
Gly-Ile-Val-His-Gln-Cys-Cys-His-Ser-Ile-Cys-Ser-
Leu-Tyr-Gln-Leu-Glu-Asn-Tyr-Cys-Asn
(His^{A4}, His^{A8}, Gly^{A21} substitutions)
SEQ. ID. NO. 6
Gly-Ile-Val-His-Gln-Cys-Cys-His-Ser-Ile-Cys-Ser-
Leu-Tyr-Gln-Leu-Glu-Asn-Tyr-Cys-Gly
(His^{B1} B-chain)
SEQ. ID. NO. 7
His-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr

Receptor-Binding Assays—Relative activity is defined as the ratio of dissociation constants pertaining to the wild-type and variant hormone-receptor complex. Data were corrected for nonspecific binding (amount of radioactivity remaining membrane associated in the presence of 1 μM human insulin). In all assays, the percentage of tracer bound in the absence of competing ligand was less than 15% to avoid ligand-depletion artifacts. Relative affinities of insulin analogues for the isolated insulin holoreceptor (isoform B) were performed using a microtiter plate antibody capture technique as known in the art. Microtiter strip plates (Nunc Maxisorb) were incubated overnight at 4° C. with AU5 IgG (100 μl/well of 40 mg/ml in phosphate-buffered saline). Binding data were analyzed by a two-site sequential model. A corresponding microtiter plate antibody assay using the IGF Type I receptor was employed to assess cross-binding to this homologous receptor.

Rodent Assay—Male Lewis rats (mean body mass ˜300 g) were rendered diabetic by streptozotocin. Effects of insulin analogs on blood glucose concentration following subcutaneous injection were assessed using a clinical glucometer (Hypoguard Advance Micro-Draw meter) in relation to wild-type insulin or buffer alone (16 mg glycerin, 1.6 mg meta-cresol, 0.65 mg phenol, and 3.8 mg sodium phosphate (pH 7.4); Lilly diluent). Wild-type insulin and [His^{A4}, His^{A8}]-insulin were made zinc-free in the above buffer. [His^{A4}, His^{A8}]-insulin and insulin glargine were also dissolved in dilute HC (pH 4) containing a 5.2:1 ratio of ZnCl₂:insulin monomer, 25 mM meta-cresol, and 185 mM glycerol. Rats were injected subcutaneously at time t=0 with 3.44 nmoles of insulin or insulin analogs (˜12-13.7 nmoles) in 100 μl of buffer per rat (for wild-type insulin this corresponds to 2 IU/kg body weight). For neutral zinc-free formulations, blood was obtained from clipped tip of the tail at time 0 and every 10 min up to 90 min. For acidic zinc formulations, blood was obtained at times 0, 1, 2, 4, 6, 10.8, and 24 h.

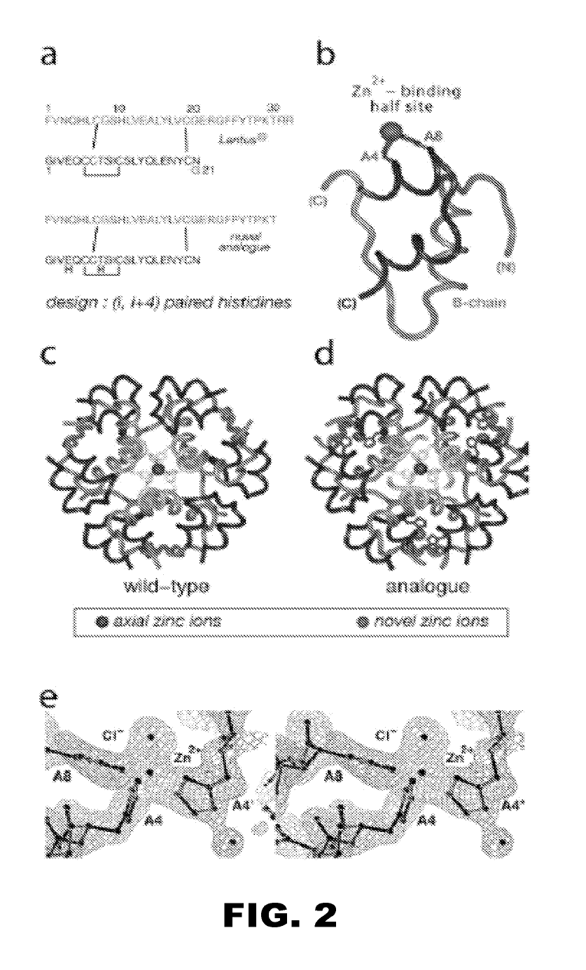

The crystal structure of [His^{A4}, His^{A8}]-insulin was determined as described below to count and visualize the number of zinc ions per hexamer and to test whether the paired Histidine substitutions at positions A4 and A8 would direct the binding of interfacial zinc ions between hexamers in the crystal lattice. Crystals were grown in the presence of zinc ions and phenol to yield T₃R³ hexamers. The structure was obtained by molecular replacement at a resolution of 1.9 Å (Table 3). The analog's mode of hexamer assembly (FIG. 2d) is identical to that of wild-type insulin (FIG. 2c). The respective conformations of T and R^{f} protomers are essentially identical to those of wild-type insulin. No transmitted perturbations occur at proposed receptor-binding surfaces.

Wild-type and variant hexamers each contain two axial Zn ions, one per T₃ and R^{f}₃ trimer (central spheres overlaid in FIGS. 2c, 2d). Coordination at each site is mediated by trimer-related His^{B10} side chains with distorted tetrahedral geometry (light gray at center of hexamers in FIG. 2c,d). In the R^{f}₃ trimer the fourth ligand is a chloride ion whereas in the T₃ trimer this site (more exposed than in the R^{f}₃ trimer) exhibits partial occupancy by either a chloride ion or bound water molecule. These features are consistent with wild-type structures. As is also observed in wild-type crystals grown under similar conditions, the R^{f}₃ trimer contains three bound phenol molecules (not shown). The A4 and A8 substitutions thus do not block the TR transition, a classical model for the reorganization of insulin on receptor binding.

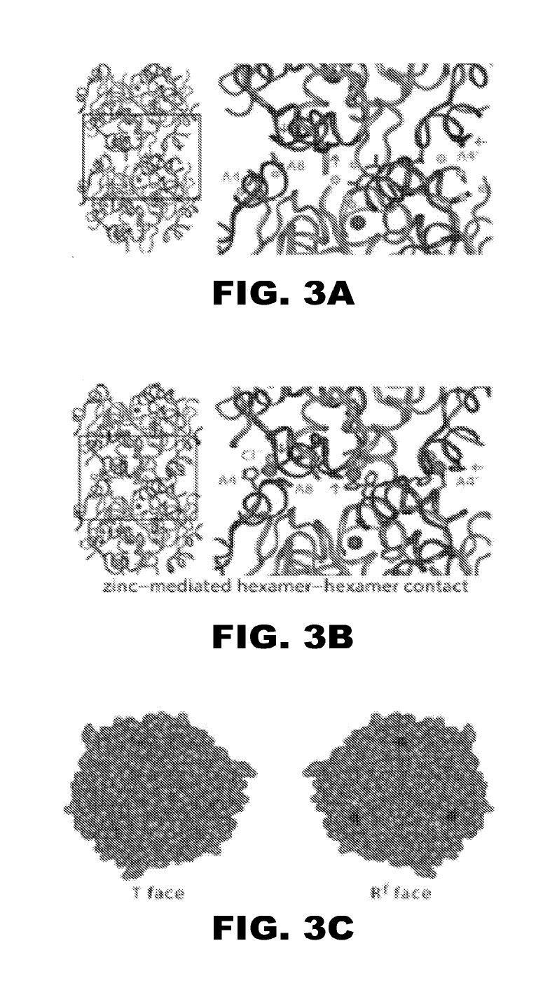

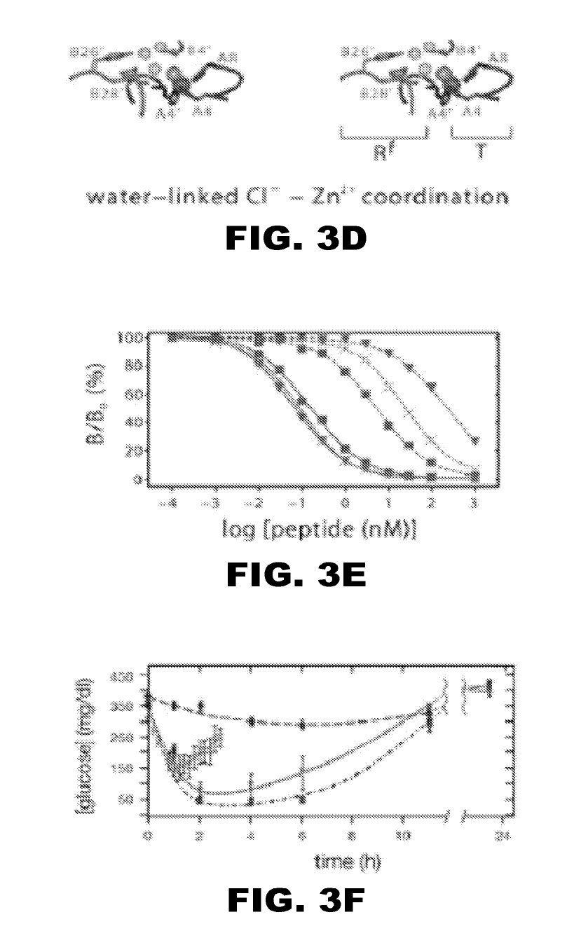

The variant T₃R^{f}₃ hexamer contains three additional trimer-related Zn ions at the T-state surfaces (magenta spheres in FIGS. 2b and 2d). These novel Zn ions are coordinated in part by His^{A4} and His^{A8} at an interfacial site. Representative electron density at the peripheral Zn-binding site defines a distorted tetrahedral site (FIG. 2e). Coordination is completed by a chloride ion and a “stapled” His^{A4} side chain belonging to an R^{f} protomer of an adjoining hexamer (labeled A4′ in FIG. 2e and brown arrows in FIG. 3b). Views of the opposing T and R^{f} faces of adjoining hexamers are shown in FIG. 3c (90° rotated from the orientation shown in FIG. 3b). Binding of the chloride ion is also stabilized by a network of three water molecules bound to the R^{f} protomer (smaller spheres in stereo pairs in FIG. 3d); His^{A8} in R^{f} is displaced from the zinc-binding site. The three non-classical Zn ions thus bridge the T₃ and R^{f}₃ trimers of adjacent hexamers in the lattice (larger spheres, FIGS. 3b and 3d), in part displacing water molecules ordinarily bound at the wild-type interface (smaller spheres in FIG. 3a). N—Zn²⁺ bond distances and angles are similar to those of the axial metal-ion-binding sites. The side-chain conformations of His^{A4} and His^{A8} differ between T and R^{f} protomers.

Studies of hormone binding to IR and IGF-1R were undertaken to assess relative affinities and receptor-binding selectivity (FIG. 3e and Table 2). Ligands were characterized as zinc-free monomers. Relative to the binding of human insulin to IR and IGF-IR (solid and dotted lines with crosses marking data points in FIG. 3e, respectively), insulin glargine (solid and dotted lines with squares marking data points) exhibits 2-fold reduced affinity for IR and 3-fold enhanced affinity for IGF-1R. By contrast [His^{A4}, His^{A8}]-insulin exhibits native-like affinity (solid line, inverted triangles in FIG. 3e) for IR but 6-fold reduced affinity for IGF-1R (dotted line inverted triangles, shifted to right). Thus, whereas the receptor-binding selectivity of insulin glargine is impaired by ca. 6-fold, that of [His^{A4}, His^{A8}]-insulin is enhanced by 7.5(±2.5)-fold. This represents an improvement of at least 30-fold relative to insulin glargine.

The potency and duration of action of [His^{A4}, His^{A8}]-insulin were tested in streptozotocin-induced diabetic rats in relation to insulin glargine (FIG. 3f). Glycemic control by long-acting insulin analogs in rodents (5-10 h) is less prolonged than in humans (18-24 h), presumably due to the smaller size of subcutaneous depot. [His^{A4}, His^{A8}]-insulin and insulin glargine were dissolved (like Lantus®) in dilute HCl (pH 4.0) with a molar Zn²⁺:insulin ratio of 5.2:1. The time course and extent of glycemic control were similar on injection of the two analogs (dotted and dotted/dashed lines in FIG. 3f). A rapid-acting control was provided by zinc-free human insulin in Lilly diluent (line ending at about 3 hours in FIG. 3f). Because the rats ate only at night, effects of daytime insulin injections were influenced by diurnal fasting; controls were provided by injection of diluent alone (dashed line in FIG. 30. Control studies were also undertaken of [His^{A4}, His^{A8}]-insulin in zinc-free neutral Lilly diluent; its time course was similar to that of wild-type insulin control (not shown) Zinc-free glargine was not tested at neutral pH due to its sparing solubility.

X-Ray Crystallography

Crystals were grown by hanging-drop vapor diffusion in the presence of a 1:1.7 ratio of Zn²⁺ to protein monomer and a 3.5:1 ratio of phenol to protein monomer in Tris-HCl buffer. Drops consisted of 1 μl of protein solution (8 mg/ml in 0.02 M HCl) mixed with 1 ml of reservoir solution (0.38 M Tris-HCl, 0.1 M sodium citrate, 9% acetone, 4.83 mM phenol, and 0.8 mM zinc acetate at pH 8.4). Each drop was suspended over 1 ml of reservoir solution. Crystals were obtained at room temperature after two weeks. Data were collected from single crystals mounted in a rayon loop and flash frozen to 100° K. Reflections from 32.05-1.90 Å were measured with a CCD detector system on synchrotron radiation in Berkeley National Laboratory. Data were processed with the program DTREK. The crystal belongs to space group R3 with unit cell parameters: a=b=78.09 Å, c=36.40 Å, α=β=90°, γ=120°. The structure was determined by molecular replacement using CNS. Accordingly, a model was obtained using the native TR dimer (Protein Databank (PDB) identifier 1RWE following removal of all water molecules, zinc- and chloride ions). A translation-function search was performed using coordinates from the best solution for the rotation function following analysis of data between 15.0 and 4.0 Å resolutions. Rigid-body refinement using CNS, employing overall anisotropic temperature factors and bulk-solvent correction, yielded values of 0.325 and 0.344 for R and R_{free}, respectively, for data between 19.2 and 3.0 Å resolution. Between refinement cycles, 2Fₒ-F_{c} and Fₒ-F_{c} maps were calculated using data to 3.0 Å resolution; zinc and chloride ions and phenol molecules were built into the structure using the program O (Jones et al., Acta Crystallogr. A., Vol. 4, pp. 110-119 (1991)). Water molecules were calculated and checked using DDQ program (Focco Van Akker and Wim Hol, Acta Cryst. 1999, D55, 206-218). The geometry was continually monitored with PROCHECK (Laskowski et al., J. Appl. Crystallogr., Vol. 26, pp. 283-291 (1993)); zinc ions and water molecules were built into the difference map as the refinement proceeded. Calculation of omit maps (especially in the first eight residues of B chain N terminus of each monomer) and further refinement were carried out using CNS, which implement maximum-likelihood torsion-angle dynamics and conjugate-gradient refinement.

TABLE 3
X-ray data collection and refinement statistics

| | [His^{A4}, His^{A8}]-insulin |
|---|---|
| Data collection | |
| Space group | R3 |
| Cell dimensions | |
| a, b, c (Å) | 78.09, 78.09, 36.40 |
| α, β, γ (°) | 90.00, 90.00, 120.00 |
| Resolution (Å) | 32.05-1.90 |
| R_{sym} or R_{merge} | 0.057(0.422)* |
| I/σI | 14.1(3.0)* |
| Completeness (%) | 99.5(100.0)* |
| Redundancy | 5.49(5.43)* |
| Refinement | |
| Resolution (Å) | 32.05-1.90 |
| No. reflections | 6475/955 |
| R_{work}/R_{free} | 0.199/0.257 |
| No. atoms | |
| Protein | 818 |
| Ligand/ion | 6 |
| Water | 82 |
| B-factors | |
| Protein | 42.28 |
| Ligand/ion | 29.03 |
| Water | 53.94 |
| R.m.s deviations | |
| Bond lengths (Å) | 0.008 |
| Bond angles (°) | 1.2 |

*Highest resolution shell

The pH-dependent solubility of the insulin analogues was evaluated by a modification of the method of DiMarchi and coworkers (Kohn, W. D., Micanovic, R., Myers, S. L., Vick, A. M., Kahl, S. D., Zhang, L., Strifler, B. A., Li, S., Shang, J., Beals, J. M., Mayer, J. P., and DiMarchi, R. D. Peptides 28, 935-48 (2007)). In brief, wild-type human insulin, insulin glargine or [His^{A4}, His^{A8}]-insulin were made 0.60 mM in an unbuffered solution containing dilute HCl at pH 4.0; the composition of the solution, similar to that employed in the pharmaceutical formulation Lantus (Sanofi-Aventis), contained 0.52 mM ZnCl₂, 20 mg/ml of an 85% vol/vol glycerol solution (to a final concentration of 185 mM), and 2.7 mg/ml meta-cresol (25 mM) as antimicrobial preservative. Each of the three proteins exhibits a solubility in this pH 4.0 solution exceeding 0.60 mM. A series of identical aliquots (10 ml) was removed and diluted 50-fold into buffers at various pH values (in the range 5.0-9.0) to a final volume of 500 ml; respective pH values were then re-adjusted to be 5.0, 6.0, 7.4, 8.0, 8.5, and 9.0. The diluent was composed of 10 mM Tris-HCl and 140 mM NaCl with pH values adjusted by dilute HCl or NaOH. The multiple samples were then mixed 20 times by inversion and centrifuged for 5 min at 14,000 rpm in a micro-centrifuge. 200 μl of supernatant was then removed in duplicate from each tube and injected onto an analytical reverse-phase HPLC (C4 column; 25 cm×0.46 cm) with an elution gradient of acetonitrile containing 0.1% trifluoroacetic acid. In each case a single elution peak was observed, and its area quantified by integration using vendor software (Waters, Inc.). The wild-type insulin values at pH 7.4-9.0 provided a control for losses unrelated to solubility; percent recoveries were typically in the range 85-90%. The solubility of insulin glargine at pH 7.4 was found to be between 1 and 2 μM in accord with the results of DiMarchi and coworkers. This limited solubility was similar at molar ratios of zinc-to-analog of 5.2:6 (i.e., 5.2 zinc ions per hexamer) and 2.2:6 (2.2 zinc ions per hexamer), consistent with an axial role of zinc ion in the glargine hexamer. The solubility of [His^{A4}, His^{A8}]-insulin at pH 7.4 was also found to be 1-2 μM at a molar ratio of 5.2 zinc ions per hexamer.

The formulation of the present invention provides an intermediate-acting insulin analogue also containing the Lys^{B28} and Pro^{B29} of lispro insulin (Humalog®) that is easily formulated as a clear solution at pH 4 with zinc ions and phenol. Representative binding studies of an insulin analogue containing the lispro and Histidine substitutions at positions A4 and A8 (HisA4, A8 KP-ins) and wild type human insulin (HI) are provided in Table 4 in relation to Human Insulin Receptor Isoform A (HIRA), Human Insulin Receptor Isoform B (HIRA) and Insulin-like Growth Factor Receptor (IGF-1R). As seen in Table 4, HisA4, A8 KP-ins has a similar affinity for HIRA and HIRB as HI, but a greatly reduced (greater than 4-fold reduced) affinity for IGF-1R in comparison to HI.

TABLE 4

| | HIRA | | HIRB | | IGF-1R | |
|---|---|---|---|---|---|---|
| | K_{d} (nM) | SEM | K_{d} (nM) | SEM | K_{d} (nM) | SEM |
| HisA4, A8 KP-ins | 0.016 | 0.003 | 0.033 | 0.005 | 65.3 | 12.3 |
| HI | 0.023 | 0.004 | 0.062 | 0.009 | 13.2 | 2.0 |

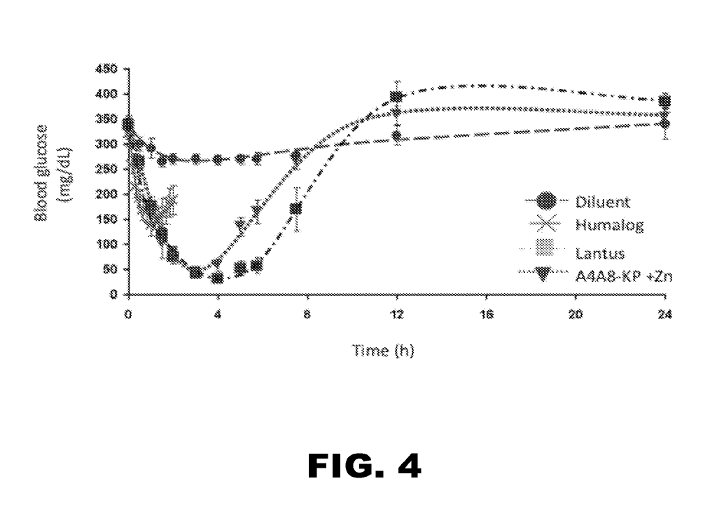

FIG. 4 provides a time course of blood glucose levels of diabetic male rats under conditions as recited with FIG. 3f. [His^{A4}, His^{A8}]-KP insulin, lispro insulin and insulin glargine were dissolved (like Lantus®) in dilute HCl (pH 4.0) with a molar Zn²⁺:insulin ratio of 5.2:1. The time course of glycemic control for [His^{A4}, His^{A8}]-KP insulin was shorter than for insulin glargine (Lantus®), but longer than for lispro insulin (Humalog®), indicating that this formulation provides an intermediate-acting insulin analogue formulation. Furthermore, crystals of HisA4, A8 KP-ins have also been obtained under similar conditions as those provided above. While not wishing to be bound by theory, it is believed that the hexamer-destabilizing effects of the lispro substitutions differ from, and are at least partially offset by, the hexamer complex-stabilizing effects of the [His^{A4}, His^{A8}] substitutions, resulting in an intermediate-acting analogue.

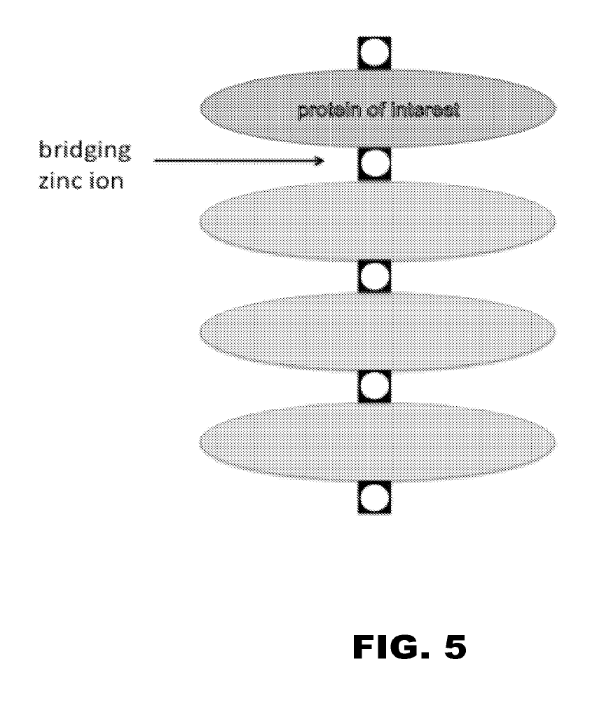

It is also envisioned that [His^{A4}, His^{A8}] insulin analogues may also contain other substitutions, such as Asp^{B28}, to obtain other intermediate-acting insulin analogue formulations. It is further envisioned that the incorporation of paired zinc-coordinating amino acid side chains, such as Histidine side chains, on the surface of a protein's structure, may be utilized in other proteins (FIG. 5) to stabilize higher order structures, such as protein hexamers, as in insulin. More particularly, we envisage that side-chains from paired Histidine substitutions in alpha-helix-containing proteins can coordinate with complementary side-chains in other polymers to create multi-polymer complexes. Examples of alpha-helix containing proteins of therapeutic utility are erythropoietin and mammalian growth hormones

Based upon the foregoing disclosure, it should now be apparent that the insulin analogues containing a combination of A-chain substitutions as provided herein will provide long-acting duration of insulin action when formulated in the presence of zinc ions and will concomitantly exhibit decreased absolute and relative affinity for the Type I IGF receptor while retaining at least 20% of the affinity of human insulin for the insulin receptor.

SEQUENCES
(proinsulin)
SEQ. ID. NO. 1
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg-Glu-Ala-Glu-Asp-
Leu-Gln-Val-Gly-Gln-Val-Glu-Leu-Gly-Gly-Gly-Pro-
Gly-Ala-Gly-Ser-Leu-Gln-Pro-Leu-Ala-Leu-Glu-Gly-
Ser-Leu-Gln-Lys-Arg-Gly-Ile-Val-Glu-Gln-Cys-Cys-
Thr-Ser-Ile-Cys-Ser-Leu-Tyr-Gln-Leu-Glu-Asn-Tyr-
Cys-Asn
(A-chain)
SEQ. ID. NO. 2
Gly-Ile-Val-Glu-Gln-Cys-Cys-Thr-Ser-Ile-Cys-Ser-
Leu-Tyr-Gln-Leu-Glu-Asn-Tyr-Cys-Asn
(B-chain)
SEQ. ID. NO. 3
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr
(paired Histidine substitutions at A4 and A8)
SEQ. ID. NO. 4
Gly-Ile-Val-His-Gln-Cys-Cys-His-Ser-Ile-Cys-Ser-
Leu-Tyr-Gln-Leu-Glu-Asn-Tyr-Cys-Xaa
Xaa = Asn, Gly, Ala, Ser, Thr
(His^{A4}, His^{A8} substitutions)
SEQ. ID. NO. 5
Gly-Ile-Val-His-Gln-Cys-Cys-His-Ser-Ile-Cys-Ser-
Leu-Tyr-Gln-Leu-Glu-Asn-Tyr-Cys-Asn
(His^{A4}, His^{A8}, Gly^{A21} substitutions)
SEQ. ID. NO. 6
Gly-Ile-Val-His-Gln-Cys-Cys-His-Ser-Ile-Cys-Ser-
Leu-Tyr-Gln-Leu-Glu-Asn-Tyr-Cys-Gly
(His^{B1} B-chain)
SEQ. ID. NO. 7
His-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr
(lispro-B-chain)
SEQ. ID. NO. 8
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Lys-Pro-Thr
(aspart-B-chain)
SEQ. ID. NO. 9
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Asp-Lys-Thr
(AspB10-B-chain)
SEQ. ID. NO. 10
Phe-Val-Asn-Gln-His-Leu-Cys-Gly-Ser-Asp-Leu-Val-
Glu-Ala-Leu-Tyr-Leu-Val-Cys-Gly-Glu-Arg-Gly-Phe-
Phe-Tyr-Thr-Pro-Lys-Thr
(DKP B-Chain Sequence)
SEQ. ID. NO. 11
Phe-Val-Asn-Gln-His-Leu-Cys-Gly-Ser-Asp-Leu-Val-
Glu-Ala-Leu-Tyr-Leu-Val-Cys-Gly-Glu-Arg-Gly-Phe-
Phe-Tyr-Thr-Lys-Pro-Thr
(Glargine B-chain)
SEQ. ID. NO. 12
Phe-Val-Asn-Gln-His-Leu-Cys-Gly-Ser-His-Leu-Val-
Glu-Ala-Leu-Tyr-Leu-Val-Cys-Gly-Glu-Arg-Gly-Phe-
Phe-Tyr-Thr-Pro-Lys-Thr-Arg-Arg
(Glargine A-chain)
SEQ. ID. NO 13
Gly-Ile-Val-Glu-Gln-Cys-Cys-Thr-Ser-Ile-Cys-Ser-
Leu-Tyr-Gln-Leu-Glu-Asn-Tyr-Cys-Gly
(Arg^{A0}, His^{A4}, His^{A8}, Gly^{A21} substitution)
SEQ. ID. NO. 14
Arg-Gly-Ile-Val-His-Gln-Cys-Cys-His-Ser-Ile-Cys-
Ser-Leu-Tyr-Gln-Leu-Glu-Asn-Tyr-Cys-Gly

We claim:

1. A method treating a patient, the method comprising administering a pharmaceutical formulation containing a physiologically effective amount of an insulin analogue or a physiologically acceptable salt thereof to the patient, wherein the insulin analogue or a physiologically acceptable salt thereof contains an insulin A-chain sequence that contains paired Histidine substitutions at A4 and A8, and optionally a substitution at A21 selected from the group consisting of Glycine, Alanine, Serine, and Threonine, and additionally containing zinc ions at a relative concentration of at least about 4 zinc ions per 6 insulin analogue molecules.

2. The method of claim 1, wherein the insulin analogue or a physiologically acceptable salt thereof is a micro-crystalline insulin suspension at pH 6.5-7.5.

3. The method of claim 1, wherein the insulin analogue or a physiologically acceptable salt thereof is formulated as a clear unbuffered solution at pH 3.5-5 containing zinc ions at a relative concentration of 4-6 zinc ions per 6 insulin analogue molecules, a preservative selected from phenol and meta-cresol, and an excipient.

4. The method of claim 1, wherein the insulin analogue or a physiologically acceptable salt thereof is formulated as a micro-crystalline insulin suspension modified to include 4-6 zinc ions per 6 insulin analogue molecules.

5. The method of any one of claims 1-4, wherein the insulin analogue or a physiologically acceptable salt thereof is modified at position A21 by substitution with Glycine.

6. The method of any one of claims 1-4, wherein the insulin analogue or a physiologically acceptable salt thereof is modified at position A21 by substitution with Alanine, Serine, or Threonine.

7. The method of claim 6, wherein the insulin analogue or a physiologically acceptable salt thereof also is modified by extension of the B-chain to include one or two C-terminal basic residues.

8. The method of claim 6, wherein the insulin analogue or a physiologically acceptable salt thereof also is modified by extension of the B-chain to include at least one N-terminal basic residues.

9. The method of claim 6, wherein the insulin analogue or a physiologically acceptable salt thereof also is modified by extension of the A-chain to include at least one N-terminal basic residues.

10. A nucleic acid encoding an insulin A-chain polypeptide, wherein the A-chain polypeptide comprises SEQ. ID. NO. 4.

11. An expression vector comprising the nucleic acid sequence of claim 10.

12. A host cell transformed with the expression vector of claim 10.

13. A pharmaceutical formulation comprising an insulin analogue or a physiologically acceptable salt thereof, wherein the insulin analogue or a physiologically acceptable salt thereof contains an insulin A-chain sequence contains paired Histidine substitutions at A4 and A8, and optionally a substitution at A21 selected from the group consisting of Glycine, Alanine, Serine, and Threonine, in a pharmaceutically acceptable buffer containing at least about 4 zinc ions per 6 insulin analogue molecules, wherein the formulation forms a long-acting zinc-dependent subcutaneous depot.

14. The pharmaceutical formulation of claim 13, wherein the insulin analogue or a physiologically acceptable salt thereof is formulated as a micro-crystalline insulin suspension at pH 6.5-7.5.

15. The pharmaceutical formulation of claim 13, formulated as a clear unbuffered solution at pH 3.5-5, and further comprising a preservative selected from phenol and meta-cresol, and an excipient.

16. The pharmaceutical formulation of claim 13, wherein the insulin analogue or a physiologically acceptable salt thereof is formulated as a micro-crystalline insulin suspension modified to include 4-6 zinc ions per 6 insulin analogue molecules.

17. The pharmaceutical formulation of claim 14, wherein the insulin analogue or a physiologically acceptable salt thereof also is modified at position A21 by substitution with Gly.

18. The pharmaceutical formulation of claim 15, wherein the insulin analogue or a physiologically acceptable salt thereof also is modified at position A21 by substitution with Gly.

19. The pharmaceutical formulation of claim 16, wherein the insulin analogue or a physiologically acceptable salt thereof also is modified at position A21 by substitution with Gly.

20. The pharmaceutical formulation of any one of claim 14, 15, or 16, wherein the insulin analogue or a physiologically acceptable salt thereof also is modified at position A21 by substitution with Ala, Ser, or Thr.

21. The pharmaceutical formulation of any one of claim 13, 17, or 20, wherein the insulin analogue or a physiologically acceptable salt thereof also is modified by extension of the B-chain to include one or two C-terminal basic residues.

22. The pharmaceutical formulation of any one of claim 13, 17, or 20, wherein the insulin analogue or a physiologically acceptable salt thereof also is modified by extension of the B-chain to include one or two N-terminal basic residues.

23. The pharmaceutical formulation of any one of claim 13, 17, or 20, wherein the insulin analogue or a physiologically acceptable salt thereof also is modified by extension of the A-chain to include one or two N-terminal basic residues.

24. A crystalline formulation of an insulin analogue, wherein the insulin analogue contains an insulin A-chain sequence containing paired Histidine substitutions at A4 and A8, and optionally a substitution at A21, in a pharmaceutically acceptable buffer containing at least about 4 zinc ions per 6 insulin analogue molecules, wherein the formulation forms a long-acting zinc-dependent subcutaneous depot, wherein the insulin analogue forms a crystalline suspension at about pH 6-7.4.

25. The method of claim 1 where the insulin analogue forms a long-acting zinc-dependent subcutaneous depot upon subcutaneous injection.

26. A pharmaceutical formulation of any one of claim 13, 17, or 20 wherein the formulation forms a long-acting zinc-dependent subcutaneous depot upon subcutaneous injection.

27. The pharmaceutical formulation of any one of claim 13, 17, or 20 wherein the insulin analogue or a physiologically acceptable salt thereof, in a zinc-free formulation, exhibits decreased affinity for the type I Insulin-like Growth Factor receptor in comparison to wild type insulin of the same species and at least 20% of the affinity of wild-type insulin for the insulin receptor of the same species.

28. A pharmaceutical formulation comprising an insulin analogue or a physiologically acceptable salt thereof, wherein the insulin analogue or a physiologically acceptable salt thereof contains an insulin A-chain sequence contains paired Histidine substitutions at A4 and A8, a Lysine or Aspartic acid substitution at B28, optionally a Proline substitution at B29, and optionally a substitution at A21 selected from the group consisting of Glycine, Alanine, Serine, and Threonine, in a pharmaceutically acceptable buffer containing at least about 4 zinc ions per 6 insulin analogue molecules, wherein the formulation forms an intermediate-acting zinc-dependent subcutaneous depot.

29. A pharmaceutical formulation comprising an insulin analogue or a physiologically acceptable salt thereof, wherein the insulin analogue or a physiologically acceptable salt thereof contains an insulin A-chain sequence contains paired Histidine substitutions at A4 and A8, an amino acid substitution other than Cysteine, Lysine or Aspartic acid at B28, optionally a Proline substitution at B29, and optionally a substitution at A21 selected from the group consisting of Glycine, Alanine, Serine, and Threonine, in a pharmaceutically acceptable buffer containing at least about 4 zinc ions per 6 insulin analogue molecules, wherein the formulation forms an intermediate-acting zinc-dependent subcutaneous depot.

30. A pharmaceutical formulation comprising two or more insulin analogues or physiologically acceptable salts thereof, wherein at least one insulin analogue or a physiologically acceptable salt thereof contains an insulin A-chain sequence contains paired Histidine substitutions at A4 and A8, in a pharmaceutically acceptable buffer containing at least about 4 zinc ions per 6 insulin analogue molecules, wherein the formulation provides a mixed long-, intermediate-, and/or short acting zinc-dependent subcutaneous depot.


SEQUENCE LISTING (14 sequences)

SEQ ID NO: 1 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 2 - protein, 21 aa, Homo sapiens
GIVEQCCTSICSLYQLENYCN

SEQ ID NO: 3 - protein, 30 aa, Homo sapiens
FVNQHLCGSHLVEALYLVCGERGFFYTPKT

SEQ ID NO: 4 - protein, 21 aa, Homo sapiens
  MISC_FEATURE (21)..(21): Xaa = Asn, Gly, Ala, Ser, Thr
GIVHQCCHSICSLYQLENYCX

SEQ ID NO: 5 - protein, 21 aa, Homo sapiens
GIVHQCCHSICSLYQLENYCN

SEQ ID NO: 6 - protein, 21 aa, Homo sapiens
GIVHQCCHSICSLYQLENYCG

SEQ ID NO: 7 - protein, 30 aa, Homo sapiens
HVNQHLCGSHLVEALYLVCGERGFFYTPKT

SEQ ID NO: 8 - protein, 30 aa, Homo sapiens
FVNQHLCGSHLVEALYLVCGERGFFYTKPT

SEQ ID NO: 9 - protein, 30 aa, Homo sapiens
FVNQHLCGSHLVEALYLVCGERGFFYTDKT

SEQ ID NO: 10 - protein, 30 aa, Homo sapiens
FVNQHLCGSDLVEALYLVCGERGFFYTPKT

SEQ ID NO: 11 - protein, 30 aa, Homo sapiens
FVNQHLCGSDLVEALYLVCGERGFFYTKPT

SEQ ID NO: 12 - protein, 32 aa, Homo sapiens
FVNQHLCGSHLVEALYLVCGERGFFYTPKTRR

SEQ ID NO: 13 - protein, 21 aa, Homo sapiens
GIVEQCCTSICSLYQLENYCG

SEQ ID NO: 14 - protein, 22 aa, Homo sapiens
RGIVHQCCHSICSLYQLENYCG